SEQUENCE LISTING (21 sequences)

SEQ ID NO: 1 - protein, 21 aa, Homo sapiens
EFTEDELPYDDCVFGGQRLTL

SEQ ID NO: 2 - protein, 22 aa, Homo sapiens
GLDGIIEYDDFKLNSSIVEPKE

SEQ ID NO: 3 - protein, 21 aa, synthetic construct
EFTEDELPYDDLVFGGQRLTL

SEQ ID NO: 4 - protein, 21 aa, synthetic construct
EFTEDELPYDDFVFGGQRLTL

SEQ ID NO: 5 - protein, 21 aa, synthetic construct
EFTEDELPSDSCVFGGQRLTL

SEQ ID NO: 6 - protein, 21 aa, synthetic construct
EFTEDELPYDDLVFGGQKLTL

SEQ ID NO: 7 - protein, 22 aa, Homo sapiens
LSSANSLYDDIECFLMELEQPA

SEQ ID NO: 8 - protein, 51 aa, synthetic construct
NNTSSSPQPKKKPLDGEYFTLQIRGRERFEMFRELNEALELEDAQAGKEPG

SEQ ID NO: 9 - protein, 13 aa, synthetic construct
GGGGSGGGSGGGS

SEQ ID NO: 10 - protein, 5 aa, synthetic construct
  REPEAT 1..5: note = repeat unit, repeated n times wherein n is an integer inferior to 4
GGGGS

SEQ ID NO: 11 - protein, 85 aa, synthetic construct
NNTSSSPQPKKKPLDGEYFTLQIRGRERFEMFRELNEALELEDAQAGKEPGGGGGSGGGSGGGSEFTEDELPYDDLVFGGQRLTL

SEQ ID NO: 12 - protein, 21 aa, synthetic construct
EFTEDELPSDSCVFGGQKLTL

SEQ ID NO: 13 - protein, 21 aa, Homo sapiens
DSGDEGDEYDDIVVHSSRSQT

SEQ ID NO: 14 - protein, 21 aa, Homo sapiens
PGLDGIIEYDDFKLNSSIVEP

SEQ ID NO: 15 - protein, 21 aa, Homo sapiens
CLSSANSLYDDIECFLMELEQ

SEQ ID NO: 16 - protein, 17 aa, synthetic construct
DELPYDDCVFGGQRLTL

SEQ ID NO: 17 - protein, 17 aa, synthetic construct
DELPYDDIVFGGQRLTL

SEQ ID NO: 18 - protein, 17 aa, synthetic construct
DELPYDDWVFGGQRLTL

SEQ ID NO: 19 - protein, 15 aa, synthetic construct
LPYDDCVFGGQRLTL

SEQ ID NO: 20 - protein, 13 aa, synthetic construct
YDDCVFGGQRLTL

SEQ ID NO: 21 - protein, 10 aa, synthetic construct
DELPYDDCVF

(19) United States
(12) Patent Application Publication
(10) Pub. No.: US 2026/0265321 A1
(43) Pub. Date: Sep. 10, 2026

(54) PEPTIDE AND POLYPEPTIDE INHIBITING THE NF-KB SIGNALING PATHWAY

(71) Applicants: Centre National de la Recherche Scientifique, Paris (FR); Université de Strasbourg, Strasbourg (FR); Université de Liège, Liège (BE)

(72) Inventors: Katia ZANIER, Schiltigheim (FR); Mariel DONZEAU, Illkirch (FR); Alain CHARIOT, Neupré (BE); Kateryna SHOSTAK, Liège (BE); Changqing LI, llkirch-Graffenstaden (FR)

(21) Appl. No.: 19/491,340

(22) PCT Filed: Jun. 6, 2024
(86) PCT No.: PCT/EP2024/065645

§ 371 Date: Dec. 8, 2025

(30) Foreign Application Priority Data
Jun. 8, 2023 (EP) 23305915.3

(51) Int. Cl. C07K 14/47; A61K 38/00

(52) U.S. Cl. CPC C07K 14/4703; A61K 38/00; C07K 2319/21; C07K 2319/24

(57) ABSTRACT

The present invention concerns new peptides and polypeptides that bind to IKK and inhibit the NF-κB signaling pathway. The present invention also relates to a method of inhibiting the NF-κB signaling pathway and a method of identifying small molecules targeting IKK dimers. The present invention further provides pharmaceutical compositions and their use in a method for treating and/or preventing a disorder regulated by the NF-κB signaling pathway.

### CROSS-REFERENCE TO RELATED APPLICATIONS

The present application is a filing under 35 U.S.C. 371 as the National Stage of International Application No. PCT/EP2024/065645, filed Jun. 6, 2024, entitled “PEPTIDE AND POLYPEPTIDE INHIBITING THE NF-KB SIGNALING PATHWAY,” which claims priority to European Application No. 23305915.3 filed with the European Patent Office on Jun. 8, 2023, both of which are incorporated herein by reference in their entirety for all purposes.

### INCORPORATION BY REFERENCE OF MATERIAL IN XML FILE

This application incorporates by reference the Sequence Listing contained in the following XML file being submitted concurrently herewith:

File name: 4692-25400_BNT241786USPC_Sequence_Listing.xml, created on Nov. 6, 2025; and having a file size of 19.2 KB. The information in the Sequence Listing is incorporated herein in its entirety for all purposes.

The present invention concerns new peptides and polypeptides that bind to IKK and inhibit the NF-κB signaling pathway. The present invention also relates to a method of inhibiting the NF-κB signaling pathway and a method of identifying small molecules targeting IKK dimers. The present invention further provides pharmaceutical compositions and their use in a method for treating and/or preventing a disorder regulated by the NF-κB signaling pathway.

Nuclear factor κB (NF-κB) signaling is deeply involved in the regulation of cellular inflammatory, immune and apoptotic responses.

Under homeostatic conditions, NF-κB dimers are sequestered in the cytoplasm by interaction with inhibitor of κB (IκB) proteins or with the NF-κB precursor proteins p100 and p105. Receptor stimulation of the ‘canonical’ NF-κB pathway activates the IKK kinase, which in turn phosphorylates IκB proteins, thereby resulting in ubiquitin-mediated degradation of IκB and subsequent nuclear translocation of p65/p50 NF-κB dimers (see FIG. 12). Stimulation of the ‘alternative’ NF-κB pathway leads instead to IKK-mediated phosphorylation of p100, which triggers partial degradation of p100 into the mature p52 NF-κB subunit.

Different IKK complexes (collectively referred to as IKKs hereafter) exist in cells, which exhibit variable subunit composition. The canonical complex is the most abundant IKK species in cells in the stimulated condition. The core of the canonical IKK complex comprises a heterodimer of the catalytic IKKα and IKKβ subunits and two copies of the regulatory NEMO subunit. IKKα is necessary for IKKβ activation through phosphorylation of its activation loop. In contrast, the core complex acting in the alternative pathway is NEMO independent and consists of a homodimer of IKKα (see FIG. 12).

Besides the roles in NF-κB signaling, IKKα and IKKβ target a variety of substrates involved in key cellular functions. In particular, IKKα homodimers play important roles in transcription by phosphorylating transcription factors (eg. IRF7), co-activators and co-repressors (eg. SMRT, CBP, SRC-3), and histone H3. IKKβ has instead been shown to regulate insulin signaling by phosphorylating IRS-1 and to act on several tumor suppressor pathways (eg. FOXO3a, TSC1 and p53).

Deregulation of NF-κB signaling pathway occurs in many pathologies, including infectious, inflammatory, autoimmune diseases, and cancer. Several enzymatic inhibitors of IKKs have thus been developed, with most of them targeting the IKKβ catalytic subunit (ATP analogues, allosteric inhibitors and thiol-reactive compounds). However, none of these inhibitors are used in the clinic due to the severe side effects, which are linked to
(i) the off-target effects due to the structural similarity of the kinase domain,
(ii) the multiple cellular functions of IKKs, including NF-κB-independent functions and
(iii) the crucial role of NF-κB signaling in innate immunity, whose systemic inhibition is responsible for severe immunodeficiencies.
   On top of this, additional concerns on systemic IKK inhibition are related to the fact that IKK/NF-κB act as a “double-edged sword”, having pro- and anti-inflammatory functions in several disease contexts.

NEMO-based approaches have also been developed. Cell permeable peptides corresponding to the NEMO binding domain of IKKα and IKKβ were shown to inhibit indirectly canonical IKK activity through blockade of the essential interaction with NEMO. These peptides have been shown to ameliorate disease symptoms in mouse models of inflammatory bowel disease. Peptides and small molecules targeting the ubiquitin binding region of NEMO have been proposed for therapeutic applications (patent EP 1677815). However, a major caveat of the NEMO-based approaches is that, similarly to the IKKβ enzymatic inhibitors, they target the canonical NF-κB pathway only, while it is well established that deregulation of alternative NF-κB signaling regulated by IKKα homodimers is crucial for inflammatory and autoimmune diseases, and in both solid and hematological cancers.

For all these reasons, it is important to develop new drugs, which are more selective for the NF-κB pathways compared to the existing enzymatic inhibitors of IKKβ and, which are able to tone down the NF-κB response (i.e. to act as modulators) without completely suppressing it.

### SUMMARY OF THE INVENTION

The inventors have identified a novel peptide with a sequence having a new docking ground, which mediates IKK interactions with key NF-κB substrates, namely IκBα and IκBβ of the canonical pathway and p100 of the alternative pathway, thus unveiling new possibilities for the rational design of therapeutic inhibitors of IKK. This peptide is very specific to NF-κB pathway thus limiting off-target effects.

In a first aspect, the invention provides an isolated peptide, preferably having 10 to 35 amino acids, comprising the amino acid sequence YDDφ₁Xφ₂, wherein φ₁ and φ₂ are hydrophobic amino acids and X is any amino acid.

Also provided is a polypeptide fusion construct comprising a tag fused to the N-terminal end of a peptide according to the invention.

In a second aspect, the invention concerns a method of inhibiting the NF-κB signaling pathway comprising contacting in vitro IKKα and/or IKKβ with a peptide according to the invention or a polypeptide fusion construct according to the invention.

The invention also concerns a method of inhibiting the NF-κB signaling pathway comprising contacting in vitro or in cellulo a polypeptide fusion construct according to the invention. “In cellulo” is used to refer to observations made in vitro in isolated living cells, and “in vitro” is used for this purpose but also for cell-free systems (e.g. an enzyme assay in a test tube versus the same assay performed with intact cells). By contrast, “in vivo” is always used to refer to observations made in whole organisms.

The invention provides a pharmaceutical composition comprising a peptide according to the invention, or a polypeptide fusion construct according the invention.

The invention also provides for a pharmaceutical composition for use in a method for treating and/or preventing a disorder, preferably regulated by the NF-κB signaling pathway, in a subject in need thereof, preferably wherein said disorder is linked to an hyperactivation of the NF-κB signaling pathway, or wherein said disorder is selected from the group consisting of inflammatory diseases, oncogenesis, autoimmune diseases and viral infection.

In a further aspect, the invention concerns a method of identifying small molecules targeting the docking groove of IKK dimers, the method comprising contacting IKK dimers with a candidate small molecule and a peptide according to the invention or a polypeptide fusion construct according to the invention, optionally wherein the peptide or the polypeptide fusion construct further comprise a peptide tag.

The invention also relates to an isolated nucleic acid encoding a peptide according to the invention or a polypeptide fusion construct according to the invention.

The invention concerns a vector comprising a nucleic acid according to the invention.

The invention also provides for a host cell comprising a nucleic acid according to the invention or a vector according to the invention.

### DETAILED DESCRIPTION OF THE INVENTION

### Definitions

In the context of the invention, the term “peptide” refers to native peptides (either proteolysis products or synthetically synthesized peptides) and further to peptidomimetics, such as peptoids and semipeptoids which are peptide analogs, which may have, for example, modifications rendering the peptides more stable while in a body, or more immunogenic. Such modifications include, but are not limited to, cyclization, N-terminus modification, C-terminus modification, acylation, PEGylation, peptide bond modification, including, but not limited to, CH₂—NH, CH₂—S, CH₂—S═O, O═C—NH, CH₂—O, CH₂—CH₂, S═C—NH, CH═CH or CF═CH, backbone modification and residue modification. Methods for preparing peptidomimetic compounds are well known in the art and are specified in Quantitative Drug Design, CA. Ramsden Gd., Chapter 17.2, F. Choplin Pergamon Press (1992).

As used herein, the term “amino acid” is understood to include: the 20 naturally occurring amino acids i.e. alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine; amino acids harbouring the post-translational modifications which can be found in vivo such as hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

By “hydrophobic amino acid” is meant herein amino acids that have hydrophobic side chains. Hydrophobic amino acids include glycine (Gly), alanine (Ala), valine (Val), leucine (Leu), isoleucine (Ile), proline (Pro), phenylalanine (Phe), methionine (Met), cysteine (Cys), tyrosine (Tyr) and tryptophan (Trp).

The expression “nucleic acid molecule encoding” or “nucleic acid encoding” as used in the present invention refers to a nucleic acid which directs the expression of a specific protein or peptide. The term includes both the DNA molecule that is transcribed into RNA and the RNA molecule that is translated into protein. It includes both full-length nucleic acid molecules as well as shorter molecules derived from the full-length molecules. It is understood that a particular nucleic acid molecule includes the degenerate codons of the native sequence or sequences which may be introduced to provide codon preference in a specific host cell.

By “isolated”, it is meant, when referring to a peptide or a polypeptide or a nucleotide sequence, that the indicated molecule is removed from its original environment. For example, a naturally-occurring polypeptide is isolated if it is separated from some or all of the coexisting materials in the natural system.

Preferably, peptide or polypeptides provided herein are isolated to a purity of at least 80% by weight, more preferably to a purity of at least 95% by weight, and most preferably to a purity of at least 99% by weight. In general, such purification may be achieved using, for example, the standard techniques of ammonium sulfate fractionation, SDS-PAGE electrophoresis, and affinity chromatography.

A polynucleotide is considered to be isolated if, for example, it is cloned into a vector that is not a part of the natural environment. An “isolated” nucleic acid molecule that encodes a particular polypeptide refers to a nucleic acid molecule that is substantially free of other nucleic acid molecules that do not encode the subject polypeptide; however, the molecule may include some additional bases or moieties, which do not deleteriously affect the basic characteristics of the composition.

The term “linker” as used in the present invention refers to a peptide adapted to connect two peptides. The linker may contain any amino acids, the amino acids glycine (G) and serine(S) being preferred.

Such linker are well known to the skilled in the art and include for example GGGGSGGGSGGGS (SEQ ID NO: 9) or (GGGGS)n (SEQ ID NO: 10), wherein n is an integer inferior to 4.

The term “host cell” as used in the present invention refers to a prokaryotic or eukaryotic cell. Said host cells can contain an expression vector. This term is also meant to include those prokaryotic or eukaryotic cells that have been genetically engineered to contain the cloned gene(s) in the chromosome or genome of the host cell. The host cell is not limited to a unicellular organism such as E. coli and yeast. Cells from multicellular organisms such as mammals, insects, and plants are also contemplated as host cells in the context of this invention.

By “IKK dimers”, it is meant homodimers of IKKα and/or homodimers of IKKβ and/or heterodimers of IKKα/β. IKKα and IKKβ kinases are well-known to the skilled in the art. For example amino acid sequence of human IKKα and IKKβ kinases have been determined and analyzed (see for example under UniprotKB accession number O15111 and O14920 respectively as available on May 25, 2023).

By “the NF-κB signaling pathway”, it is meant the canonical and/or alternative pathway. The ‘canonical’ NF-κB pathway comprises the activation of IKK kinase, which in turn phosphorylates IκB proteins, thereby resulting in ubiquitin-mediated degradation of IκB and subsequent nuclear translocation of p65/p50 NF-κB dimers. Alternative NF-κB pathway leads to IKK-mediated phosphorylation of p100, which triggers partial degradation of p100 into the mature p52 NF-κB subunit (see FIG. 12).

The term “pharmaceutically acceptable” refers to properties and/or substances which are acceptable for administration to a subject from a pharmacological or toxicological point of view. Further “pharmaceutically acceptable” refers to factors such as formulation, stability, patient acceptance and bioavailability which will be known to a manufacturing pharmaceutical chemist from a physical/chemical point of view.

As used herein, “pharmaceutically acceptable excipient” refers to any substance in a pharmaceutical composition different from the active ingredient. Said excipients can be liquids, sterile, as for example water and oils, including those of origin in the petrol, animal, vegetable or synthetic, as peanut oil, soy oil, mineral oil, sesame oil, and similar, disintegrate, wetting agents, solubilizing agents, antioxidant, antimicrobial agents, isotonic agents, stabilizing agents or diluents. Suitable adjuvants and/or pharmaceutical carriers are described in “Remington's Pharmaceutical Sciences” by E. W. Martin.

By “subject” is meant herein a mammal, such as a rodent, a feline, a canine, or a primate. Preferably, a subject according to the invention is a human.

In the context of the invention, the term “treating” or “treatment” means reversing, alleviating, inhibiting the progress of the disorder or condition to which such term applies, or one or more symptoms of such disorder or condition.

In the context of the invention, the term “preventing” or “prevention” refers to the prophylactic treatment of a subject who is at risk of developing a condition resulting in a decrease in the probability that the subject will develop the condition or a delay in the development of the condition. It also refers to decreasing the probability that the subject having a condition to develop attacks or relapse or a delay in the development of attacks or relapse.

The term “therapeutically effective amount”, when used in reference to a pharmaceutical composition comprising one or more peptide or polypeptide fusion construct, refers to an amount or dosage sufficient to produce a desired therapeutic result. More specifically, a therapeutically effective amount is an amount of a peptide or polypeptide fusion construct sufficient to inhibit, for some period of time, one or more of the clinically defined pathological processes associated with the condition being treated. The effective amount may vary depending on the peptide or polypeptide fusion construct that is being used, and also depends on a variety of factors and conditions related to the patient being treated and the severity of the disorder. For example, if the peptide or polypeptide fusion construct is to be administered in vivo, factors such as the age, weight, and health of the patient as well as dose response curves and toxicity data obtained in preclinical animal work would be among those factors considered. The determination of an effective amount or therapeutically effective amount of a given pharmaceutical composition is well within the ability of those skilled in the art.

Throughout the instant application, the term “comprising” is to be interpreted as encompassing all specifically mentioned features as well optional, additional, unspecified ones. As used herein, the use of the term “comprising” also discloses the embodiment wherein no features other than the specifically mentioned features are present (i.e. “consisting of”).

### Peptides and Polypeptides Fusion Construct

The invention provides an isolated peptide, preferably having 10 to 35 amino acids, comprising the amino acid sequence YDDφ₁Xφ₂, wherein φ₁ and φ₂ are hydrophobic amino acids and X is any amino acid.

In a preferred embodiment, φ₁ is selected among alanine, leucine, phenylalanine isoleucine and cysteine, more preferably φ₁ is selected among leucine, phenylalanine isoleucine and cysteine. In a particularly preferred embodiment, φ₁ is leucine or phenylalanine. In another particularly preferred embodiment, φ₁ is leucine, phenylalanine, isoleucine or tryptophane.

In a preferred embodiment, X is selected among valine, lysine, arginine, isoleucine, glutamic acid and cysteine.

In a preferred embodiment, φ₂ is selected among alanine, tyrosine, leucine, phenylalanine, isoleucine, valine and cysteine, more preferably φ₂ is selected among leucine, phenylalanine, isoleucine, valine and cysteine.

The peptides of the invention preferably consist of at least 6 amino acids, at least 8 amino acids, at least 10 amino acids, at least 15 amino acids or at least 20 amino acids, and preferably consist of less than 100 amino acids, less than 50 amino acids or more preferably less than 35 amino acids. In a particular embodiment, the peptides of the invention further comprise a peptide tag as defined below.

The peptide according to the invention preferably binds to IKK dimers and preferably to homodimers of IKKα. Such binding can be evaluated by any method known to the skilled in the art, and for example by pulldown experiments, or ITC analyses, as demonstrated in the example section. The peptide according to the invention preferably binds to IKK dimers, and more preferably to homodimers of IKKα, with a K_{D} inferior to 100 μM, more preferably inferior to 50 μM.

In a particular embodiment, the peptide according to the invention comprises a sequence having at least 75%, 80%, 85% or 90% of identity with:
the amino acid sequence EFTEDELPYDDCVFGGQRLTL (SEQ ID NO: 1), or
the amino acid sequence GLDGIIEYDDFKLNSSIVEPKE (SEQ ID NO: 2).

Preferably, the peptide according to the invention has for sequence:
the amino acid sequence EFTEDELPYDDCVFGGQRLTL (SEQ ID NO: 1), or
the amino acid sequence GLDGIIEYDDFKLNSSIVEPKE (SEQ ID NO: 2).

Amino acid sequence identity is defined as the percentage of amino acid residues in the sequence that are identical with the amino acid residues in the reference sequence, after aligning the sequences and introducing gaps, if necessary, to achieve the maximum percent sequence identity, and not considering any conservative substitutions as part of the sequence identity. Sequence identity may be determined over the full length of the analysed sequence, the full length of the reference sequence, or both. The percentage of identity for protein sequences may be calculated by performing a pairwise global alignment based on the Needleman-Wunsch alignment algorithm to find the optimum alignment (including gaps) of two sequences along their entire length, for instance using Needle, and using the BLOSUM62 matrix with a gap opening penalty of 10 and a gap extension penalty of 0.5.

In a preferred embodiment, the peptide according to the invention comprises the amino acid sequence EFTEDELPYDDLVFGGQRLTL (SEQ ID NO: 3).

In an embodiment, the peptide of the invention can be modified by addition of a TAT (trans-activating transcriptional activator) peptide, a well-known peptide commonly used for the delivery of peptides and known to facilitate the in vivo administration of short peptides.

The peptide according to the invention may further comprise a peptide tag, preferably fused at its N-terminal or C-terminal end, in particular at its N-terminal end. Said peptide further comprising a tag is named herein “polypeptide fusion construct”. In consequence, the invention relates to a polypeptide fusion construct comprising a tag fused to a peptide according to the invention, preferably fused to the N-terminal end of said peptide.

The polypeptide fusion construct of the invention preferably consist of at least 30 amino acids, at least 40 amino acids, at least 50 amino acids, and preferably less than 250, 200 or 150 amino acids.

Any of a variety of art recognized peptide tags can be employed in the present invention. For example, suitable peptide tags include a: FLAG peptide, short FLAG peptide, His-6 peptide, Glutathion-S-Transferase (GST), Staphylococcal protein A, Streptococcal protein G, Calmodulin, Calmodulin binding peptides, Thioredoxin, β-galactosidase, Ubiquitin, Small Ubiquitin-like Modifier (SUMO), Chloramphenicol cetyltransferasel S-peptide (Ribonuclease A, residues 1-20), Myosin heavy chain, DsbA, Biotin subunit, Avidin, Streptavidin, Sfrp-tag, c-Myc, Dihydrofolate reductase, CKS, Polyarginine, Polycysteine, Polyphenylalanine, lac Repressor, N-terminus of the growth hormone, Maltose binding protein (MBP), E3, Galactose binding protein, Cyclomaltodextrin glucanotransferase, Callulose binding domain, Haemolysin A, TφE or TφLE, Protein kinase sites, BAI epitope, Btag, VP7 region of Bluetongue virus, Green Fluorescent Protein or any fluorochromes.

Preferably, the tag is chosen among MBP or E3. Preferably, E3 comprises a sequence having at least 75%, 80%, 85 or 90% of identity with the following sequence (SEQ ID NO: 8):

NNTSSSPQPKKKPLDGEYFTLQIRGRERFEMFRELNEALELEDAQAGKE
PG.

Thus, in an embodiment, the polypeptide fusion construct according to the invention comprises E3 as a tag, wherein the E3 is of sequence SEQ ID NO: 8.

In particular, the inventors have shown that E3 promotes dimerization of the peptide thereby enhancing inhibition of IKK dimers.

The peptide tag can also include one or more specific protease cleavage sites.

The peptide tag according to the invention may further comprise a cell penetrating sequence and/or a compound extending half-life of the peptide.

The polypeptide fusion construct may also comprise a linker between the tag and the peptide of the invention.

In a particular embodiment, the polypeptide fusion construct according to the invention comprises a sequence having at least 75%, 80%, 85 or 90% of identity with the amino acid sequence (SEQ ID NO: 11).

In particular embodiments, the peptide of the invention or the polypeptide fusion construct of the invention is modified so that its stability, in particular in vivo, and/or its circulation time is increased, compared to non-modified peptides or compared to non-modified polypeptides. Potential modifications that may be performed include PEGylation, acylation, biotinylation, acetylation, formylation, ubiquitination, amidation, enzyme labeling, or radiolabeling. For instance, varying degrees of PEGylation may be used to vary the half-life, with increased PEGylation corresponding to increased half-life. Modifications may occur at any location, including the peptidic backbone, the amino acid side chains, and the N-terminal or C-terminal end.

### Method of Inhibiting the NF-κB Signaling Pathway

The present invention also concerns a method of inhibiting the NF-κB signaling pathway comprising contacting in vitro IKKα and/or IKKβ with a peptide according to the invention or a polypeptide fusion construct according to the invention.

By “inhibiting the NF-κB signaling pathway”, it is meant inhibiting the canonical and/or the alternative pathway, and more preferably inhibiting the activation of IKK kinase thereby lowering the level of nuclear translocation of p65/p50 NF-κB dimers and/or thereby lowering the level of mature p52 NF-κB subunit.

By “contacting in vitro IKKα and/or IKKβ (or IKK dimers) with a peptide or a polypeptide fusion construct” it is meant setting in the same environment:
a) IKKα and/or IKKβ (or IKK dimers), and
b) a peptide or a polypeptide fusion construct respectively.

Preferably, such step is performed under conditions and for a time sufficient to permit interaction between a) and b).

In an embodiment according to the invention, IKKα, IKKβ are purified endogenous kinases.

In an embodiment according to the invention, IKK dimers are purified endogenous complexes.

In another embodiment, IKKα, IKKβ or IKK dimers are of recombinant origin and are purified before used for the methods of the invention.

The present invention also concerns a method of inhibiting the NF-κB signaling pathway comprising contacting in vivo or in cellulo a polypeptide fusion construct according to the invention.

In this embodiment, the NF-κB signaling pathway is inhibited in cells or in a subject. Such cells are preferably mammal cells, such as rodent, feline, canine, or primate cells. Preferably, cells according to the invention are human cells. Such cells are preferably from mesenchymal lineage and more particularly selected among: fibroblasts, sarcoma cells with epithelial morphology. They include cancer-derived cell lines that display constitutive activation of NF-κB signaling, e.g. MDA-MB231 (breast cancer), HCT116 or HT-29 (colon cancer), A549 (lung cancer), HeLa (cervical cancer), or routine laboratory cell lines such as HEK293.

By “contacting in cellulo a polypeptide fusion construct” it is meant setting in the same environment said at least one cell with said polypeptide fusion construct under conditions and for a time sufficient to permit interaction between the NF-κB signaling pathway of the cell and the polypeptide fusion construct.

By “contacting in vivo a polypeptide fusion construct” it is meant administering said polypeptide fusion construct to a subject under conditions and for a time sufficient to permit interaction between the NF-κB signaling pathway of cells of the subject and the polypeptide fusion construct.

In a preferred embodiment, the method of inhibiting the NF-κB signaling pathway comprising contacting in vivo or in cellulo a polypeptide fusion construct according to the invention, is performed with a polypeptide fusion construct wherein the tag is E3 and is fused to the N-terminal end of the peptide of the invention.

### Pharmaceutical Composition

The present invention also concerns a pharmaceutical composition comprising a peptide according to the invention or a polypeptide fusion construct according to the invention, and optionally a pharmaceutically acceptable excipient.

The pharmaceutical compositions of the invention can be formulated for a parenteral, intravenous, intradermal, intracerebroventricular, subcutaneous, intramuscular, intraperitoneal, oral (e.g., buccal, inhalation, nasal and pulmonary spray), intradermal, transdermal (topical), transmucosal or intraocular administration.

### Medical Indications

The inventors demonstrated that the affinity of the docking interaction correlates with both phosphorylation and degradation of the IκBα substrate, and that peptides incorporating the YDDφXφ docking motif are sufficient to block NF-κB signaling and in particular to block translocation of p65 into the nucleus.

Thus, the present invention also relates to a pharmaceutical composition as defined in the section “Pharmaceutical composition” above for use in a method for treating and/or preventing a disorder in a subject. Preferably said disorder is regulated by the NF-κB signaling pathway.

The present invention also concerns the use of a pharmaceutical composition as defined in the section “Pharmaceutical composition” above for the manufacture of a medicament intended to treat and/or prevent a disorder. Preferably said disorder is regulated by the NF-κB signaling pathway.

The present invention also concerns a method for treating and/or preventing a disorder in a subject, comprising administering to a subject in need thereof a therapeutically effective amount of a pharmaceutical composition as defined in the section “Pharmaceutical composition” above. Preferably said disorder is regulated by the NF-κB signaling pathway.

The peptide according to the invention or the polypeptide fusion construct according to the invention is preferably present in the pharmaceutical composition in a therapeutically effective amount.

The form of the pharmaceutical compositions, the route of administration, the dosage and the regimen naturally depend upon the condition to be treated or prevented, the severity of the illness, the age, weight, and sex of the patient, etc.

By “disorder regulated by the NF-κB signaling pathway” is meant herein disorder in which symptoms are linked to a hyperactivation or underactivation of the NF-κB signaling pathway, or wherein a hyperactivation or underactivation of the NF-κB signaling pathway has been observed. These disorders typically includes inflammatory diseases, autoimmune diseases, oncogenesis and viral infection. Preferably, disorders that can be treated and/or prevented according to the invention are disorders linked to an hyperactivation of the NF-κB signaling pathway.

By “disorders linked to an hyperactivation of the NF-κB signaling pathway” it is meant disorder in which symptoms are known to be due to a hyperactivation of the NF-κB signaling pathway, or wherein a hyperactivation of the NF-κB signaling pathway has been observed. Hyperactivation is defined by sustained transcription of NF-κB target genes even in the absence of normal physiological stimuli triggering the NF-κB pathway.

In an embodiment, the disorder regulated by the NF-κB signaling pathway is selected from the group consisting of inflammatory diseases, autoimmune diseases, oncogenesis, and viral infection, and more particularly is selected from the group consisting of rheumatoid arthritis, inflammatory bowel disease, multiple sclerosis, solid cancers such as colorectal cancer, and haematological cancers such as lymphomas and multiple myeloma.

The pharmaceutical compositions of the invention can be administered by any suitable route, in particular by parenteral, e.g., intravenous, intradermal, intracerebroventricular, subcutaneous, intramuscular, intraperitoneal, oral (e.g., buccal, inhalation, nasal and pulmonary spray), intradermal, transdermal (topical), transmucosal or intraocular route.

Thus, preferably the pharmaceutical composition of the invention is formulated for a parenteral, intravenous, intradermal, intracerebroventricular, subcutaneous, intramuscular, intraperitoneal, oral, intradermal, transdermal, transmucosal or intraocular administration.

### Methods of Identifying Small Molecules

The invention relates to a method of identifying small molecules targeting the docking groove of IKK dimers, the method comprising contacting IKK dimers with a candidate small molecule and a peptide according to the invention or a polypeptide fusion construct according to the invention.

Preferably, said method is an in vitro method.

This method will typically include a competitive binding assay or any other well-known assay.

By “small molecule”, it is meant is a molecule of low molecular weight, preferably inferior to 1000 daltons. Small molecule according to the invention are preferably organic compounds. In the context of the method of identifying small molecules targeting the docking groove of IKK dimer, small molecules tested are preferably drug candidate.

By “the docking groove of IKK dimers” it is meant herein the docking groove of the peptide of the invention on IKK dimers. As demonstrated in examples this docking groove is constituted by the two adjacent SDD domains of the IKK dimer.

Preferably, the peptide or the polypeptide fusion construct used in this method comprises a peptide tag, and/or is labeled with a fluorophore, or a radioactive tracer, thus easing the reading of the results of the method. Such fluorophore includes for example fluorescein isothiocyanate (FITC), lissamine rhodamine (LRSC) and Texas red.

By “contacting IKK dimers with a candidate small molecule and a peptide according to the invention or a polypeptide fusion construct” it is meant setting in the same environment:
a) IKK dimers,
b) a candidate small molecule, and
c) a peptide or a polypeptide fusion construct respectively.

Preferably, such step is performed under conditions and for a time sufficient to permit interaction between a), b) and/or c).

In an embodiment according to the invention, IKK dimers are purified endogenous complexes.

In another embodiment, IKKα, IKKβ or IKK dimers are of recombinant origin and are purified before used for the method of the invention.

In a preferred embodiment, the method is performed with a polypeptide fusion construct according to the invention wherein the tag is E3 and is fused to the N-terminal end of the peptide of the invention.

### Isolated Nucleic Acid

The invention also concerns an isolated nucleic acid encoding a peptide according to the invention or a polypeptide fusion construct according to invention.

The invention also relates to a vector comprising a nucleic acid according to the invention.

The term “vector” as used in the present invention is an expression vector.

The term “expression vector” refers to a DNA molecule comprising a cloned structural gene encoding a foreign protein, which provides the expression of the foreign protein in a recombinant host. Typically, the expression of the cloned gene is placed under the control of (i.e., operably linked to) certain regulatory sequences such as promoter and enhancer sequences. Promoter sequences may be either constitutive or inducible.

### Host Cell

The present invention also relates to a host cell comprising a nucleic acid as defined above or a vector as defined above.

The host cell is preferably a mammalian host cell.

The mammalian host cell may be of any mammalian origin and include, at least cells of human, bovine, canine, murine, rattus, equine, porcine, feline and/or non-human primate origin.

Preferred host cells are eukaryotic host cells, such as HEK293 and derivatives, CHO and derivatives, NS-0 and derivatives, COS-7 and derivatives or MRC5 and derivatives.

Techniques for synthesizing genes, incorporating them into host cells and expressing genes in hosts cells are well known in the art and the skilled person would readily be able to put the invention into effect using common general knowledge.

The present invention will be further illustrated by the figures and examples below.

### BRIEF DESCRIPTION OF THE SEQUENCES

| SEQ ID | | |
|---|---|---|
| NUMBER | Description | Sequence |
| 1 | IκBα pep (or P1) | EFTEDELPYDDCVFGGQRLTL |
| 2 | p100 pep | GLDGIIEYDDFKLNSSIVEPKE |
| 3 | C308L IκBα pep | EFTEDELPYDDLVFGGQRLTL |
| 4 | C308F IκBα pep | EFTEDELPYDDFVFGGQRLTL |
| 5 | YD/SS IκBα pep1 | EFTEDELPSDSCVFGGQRLTL |
| 6 | C308L/R314K IκBα | EFTEDELPYDDLVFGGQKLTL |
| | pep | |
| 7 | IRF7 pep | LSSANSLYDDIECFLMELEQPA |
| 8 | E3 | NNTSSSPQPKKKPLDGEYFTLQIRGRERFE |
| | | MFRELNEALELEDAQAGKEPG |
| 9 | linker | GGGGSGGGSGGGS |
| 10 | linker | GGGGS |
| 11 | E3-C308L IκBα pep | NNTSSSPQPKKKPLDGEYFTLQIRGRERFE |
| | fusion | MFRELNEALELEDAQAGKEPGGGGGSGG |
| | | GSGGGSEFTEDELPYDDLVFGGQRLTL |
| 12 | YD/SS IκBα pep2 | EFTEDELPSDSCVFGGQKLTL |
| 13 | IκBβ pep | DSGDEGDEYD DIVVHSSRSQT |
| 14 | p100 pep | PGLDGIIEYD DFKLNSSIVEP |
| 15 | IRF7 pep | CLSSANSLYDDIECFLMELEQ |
| 16 | IκBα 301-317 (or P2) | DELPYDDCVFGGQRLTL |
| 17 | C3081 IκBα 301-317 | DELPYDDIVFGGQRLTL |
| | (or P21) | |
| 18 | C308W IκBα 301-317 | DELPYDDWVFGGQRLTL |
| | (or P2W) | |
| 19 | IκBα 303-317 (or P3) | LPYDDCVFGGQRLTL |
| 20 | IκBα 305-317 (or P4) | YDDCVFGGQRLTL |
| 21 | IκBα 301-310 (or P6) | DELPYDDCVF |

### BRIEF DESCRIPTION OF THE FIGURES

FIG. 1: Domain architecture of IκBα. N-terminal region containing the phospho-acceptor residues (circles); ARD: ankyrin repeat domain; C-ter region containing the PEST sequence and docking site. A sequence alignment for residues 297-317 of IκBα highlights the YDDφXφ consensus.

FIG. 2: Representative dataset for the GPCA analysis of the interactions between full-length wt G1-IKKα or G1-IKKβ and G2-IκBα constructs in HEK293T cells. G2-IκBα 73-317 YD/SS: G2-IκBα(73-317) Y305S/D307S. NLR: normalized luminescence ratio. For a given protein pair A/B, NLR=(Gluc1-A+Gluc2-B)/[(Gluc1-A+Gluc2)+ (Gluc1+Gluc2-B)]. The data (mean±/−SD) are derived from triplicate measurements.

FIG. 3: Results from pulldown analyses evaluating the interactions of 6×His-IKKα(10-667) EE (upper panel) or 6×His-IKKβ(1-669) EE (lower panel) proteins with MBP-IκBα pep mutants. Three single amino acid substitutions were tested for each conserved position of the YDDφXφ consensus. IKKα or IKKβ bands were quantified and the data normalized to the interactions of IκBα pep wt to IKKα or IKKβ (100%). The data (mean±/−SD) are derived from three independent experiments.

FIG. 4: In vitro kinase activity experiments using purified full-length 6×His-IκBα proteins and endogenous IKK immunoprecipitated from HEK293T cells using an anti-NEMO antibody. Reaction were incubated at 30° C. and aliquots were taken at the indicated time points. Reactions were migrated on a 10% SDS-PAGE gel and analysed by Western blot using an anti-His antibody (IκBα), an antibody specific for Ser32/Ser36 phosphorylation (P-IκBα) and an anti-IKKβ antibody (IKKβ). Quantification of IκBα phosphorylation levels normalized to wt IκBα at 30 min (100%). The data (mean±/−SD) are obtained from three independent experiments.

FIG. 5: In cellulo degradation of retro-transduced IκBα proteins in MEF cells KO for IκBα, IκBβ, IκBε. Cells were stimulated with TNFα and collected at the indicated time points. Cellular extracts were migrated on a 10% SDS-PAGE gel and analyzed by Western blot using an antiIκBα antibody and a phospho-specific p65 antibody (P-p65). Quantification of IκBα levels normalized first to tubulin and, then, to the IκBα values at time 0 (100%). The data (mean±/−SD) are obtained from three independent experiments.

FIG. 6: In vitro inhibition of IKKβ kinase activity by synthetic IκBα pep variants. The experiment uses full-length wt 6×His-IκBα, endogenous IKK, and an excess of synthetic IκBα pep variants. The negative control peptide corresponds to the YD/SS pep1 sequence. Quantification of IκBα phosphorylation levels normalized to the values obtained in the absence of peptide (no peptide) at 30 minutes incubation (100%). The data (mean±/−SD) are obtained from three independent experiments.

FIG. 7: Schematic representation of fusion constructs used in the experiment consisting of mScarlet, the E3 dimerization peptide and the IκBα pep sequence.

FIG. 8: In vivo inhibition of NF-κB signaling by IκBα pep variants. Immunofluorescent staining of MRC5 cells electroporated with mRNAs coding for mScarlet-E3-IκBα pep fusions. The negative control peptide corresponds to the YD/SS pep1 sequence. After electroporation, cells were treated or not with TNFα (20 ng/ml) for 30 minutes before fixation. p65 was detected using an anti-p65 antibody followed by an anti-mouse Alexa-Fluor 488 (green). The mean of fluorescence intensity for each nucleus is reported for each condition. Statistical analysis was performed by the ANOVA t test on a sample of 100 cells per condition. (*) P<0.05, (**)<0.001, and n=100.

FIG. 9: (Upper panel) YDDφXφ sequences found in IκBα, IκBβ, p100 and IRF7. (Lower panel) Sequence logo calculated from the YDDφXφ sequences from IκBα, IκBβ, p100 and IRF7 (respectively SEQ ID NO: 1, 13, 14 and 15) and showing the position-specific frequency of each amino acid composing the motif.

FIG. 10: Domain architecture of p100. Disordered Nterminal region containing the YDDφXφ motif; RHD: Rel homology domain (residues 38-343) containing IKKα phosphorylation sites; ARD: ankyrin repeat domain (residues 487-758); DD: death domain (residues 764-851); Disordered C-terminal region containing IKKα phosphorylation sites.

FIG. 11: Representative dataset for the GPCA analysis of 29 the interactions between full-length wt G1-IKKα and G2-p100 constructs. G2-p100 FL YD/SS: G2-p100 FL Y15S/D17S. For a given protein pair A/B, NLR=(Gluc1-A+Gluc2-B)/[(Gluc1-A+Gluc2)+(Gluc1+Gluc2-B)].

FIG. 12: Illustration of the interactions mediated by the YDDφXφ docking motif in NF-κB signaling.

FIGS. 13A-13B: Pulldown analyses of the interactions between recombinant FIG. 13A, right panel) 6×His-IKKβ(1-669) EE or FIG. 13A, left panel; FIG. 13B) 6×His-IKKα(10-667) EE proteins, and MBP-peptide fusions of the YDDφXφ sequences reported in table above (SEQ ID NOs: 1, 13-21). IKKα and IKKβ were detected by Western blot (anti-His antibody) and MBP-peptides by Coomassie staining.

### EXAMPLES

### Example 1

### Materials and Methods

### DNA Constructs

Expression in E. coli and insect cells. Full-length and deletion constructs of IκBα for E. coli expression were amplified by PCR and cloned into the NcoI and BamH1 sites of a modified pETM-41 and/or pETM10 vectors (kindly provided by G. Stier, ZMBH, Heidelberg), allowing for expression of N-terminal MBP and 6×His tags, respectively. MBP-peptide fusions were obtained by ligation of complementary oligonucleotides coding for the peptide sequence into the NcoI and BamH1 sites of the modified pETM-41 vector. The IKKβ(1-669) S177E/S181E (i.e. IKKβ(1-669) EE) and the IKKα(10-667) S176E/S180E (IKKα(10-667) EE) constructs for baculovirus expression were amplified by PCR and inserted into a modified pBacPAK8 vector by recombinant cloning (Gateway® system, Invitrogen), thereby allowing for expression of N-terminal 6×His or Strep tags that are followed by a cleavage site for the Tobacco Etch virus (TEV) protease. Expression in mammalian cells. ORFs encoding for IKKα, IKKβ, IκBα, p100, IRF7 and constructs of these proteins used in the GPCA experiments were amplified by PCR and inserted into the pSPICA-N1 and pSPICA-N2 (both derived from the pCiNeo mammalian expression vector and kindly provided by Y. Jacob, Institut Pasteur, Paris) by Gateway cloning. These vectors enable expression of the Gluc1 (pSPICA-N1) and Gluc2 (pSPICA-N2) complementary fragments of the Gaussia princeps luciferase, which are linked to the N-terminal ends of the tested proteins by a flexible 20 amino acid linker.

### IκBα Constructs for IκBα Degradation Experiments in MEFs.

The DNA sequences coding for residues 297-317 of IκBα (i.e. IκBα pep) fused to E3 coding sequences were synthetized by Integrated DNA Technologies, and were cloned in the NheI and EcoRI sites of the pET containing the mScarlet coding sequences cloned in the NdeI-NheI sites, thereby allowing for expression of N-terminal mScarlet-E3-IκBα pep fusions. Mutagenesis. Side-directed mutagenesis of IKKα, IKKβ, IκBα, p100 and IRF7 was performed using the QuickChange II kit (Agilent).

All constructs were verified by DNA sequencing.

### Virus Production

The recombinant viruses (bacmid) for expression in insect cells were generated by cotransfection Spodoptera frugiperda Sf9 insect cells (Novagen) grown in Grace insect medium (Gibco™, #11605086) supplemented with 10 FCS (Gibco™, #26140079. Briefly, 250 ng of linearized AcMNPV BAC10:KO1629, Δv-cath/chiA, mCherry bacmid and 750 ng of pBacPAK8 vectors containing the IKKα(10-667) EE and IKKβ(1-669) EE inserts were transfected into Sf9 cells using lipofectamine 2000 (Invitrogen, #116680287) in a T25 flask. Viruses contained in the medium after one week incubation at 27° C. were subjected to a round of amplification yielding the P1 virus stock.

Viruses used for transduction of mouse embryonic fibroblasts (MEFs) were obtained by transfecting Phoenix-eco cells (5×10⁶) with 6 μg of pBABE-IkBa plasmid (empty vector was used as control) and 12 μl of Mirus as a transfection reagent. The medium was changed after 6 hours and replaced with 8 ml of fresh medium. The medium with virus was collected after 48 hours, filtered and used infect MEFs.

### Cell Culture

Spodoptera frugiperda Sf9 used to generate and amplify viruses were grown in Grace medium supplemented with 10% FCS (Gibco, #26140079) and Sf21 cells (Novagen) for expression in insect cells in the serum-free medium (Gibco, Sf-900 II SFM #10902096). HEK293T were cultured in Dulbecco's modified Eagle's medium (DMEM), supplemented with 10% FCS at 37° C. with 5% CO₂ and transfected using JetPEI® (Polyplus transfection). MEFs KO for IkBα, IkBβ, IkBε (kindly provided by Alexander Hoffmann, UCLA) were cultured in DMEM supplemented with 10% FCS and Pen-strep. Supernatants containing viruses (see above) were added to 2×10⁶ MEF cells with polybrene (4 μg/ml) overnight. MRC5 cells were maintained as monolayer in Dulbecco's modified Eagle's medium (DMEM) supplemented with 4.5 g/L glucose and 10% FCS and 1 mM pyruvate.

### Protein Expression

E. coli. MBP-IκBα, MBP-peptide and 6×His-IκBα fusion constructs for pulldown and in vitro activity assays were expressed in E. coli BL21 (DE3) cells. Cultures were grown at 37° C. until an optical density of 0.2-0.6 at 600 nm. Hence, cultures were incubated overnight at 18° C. Cells were harvested and flash frozen in liquid nitrogen.

Insect cells. For production of 6×His-IKKα(10-667) EE and 6×His-IKKβ(1-669) EE homodimer samples, Sf21 cells grown in suspension were infected with the corresponding virus, harvested after 3 days incubation at 27° C., washed in PBS containing 10% v/v glycerol and flash frozen in liquid nitrogen. Typically, cells are grown in 2 L flasks containing 500 ml of medium and are infected at a density of 1·10⁶ cells/ml using 20 ml of P1 virus stock (assuming a titer of 5·10⁷ pfu/ml, it corresponds to a multiplicity of infection of 2). For production of the IKKα/b heterodimer, Sf21 cells were co-infected with Strep-IKKα(10-667)

EE and 6×His-IKKβ(1-669) EE viruses. Cells were typically infected at a density of 2·10⁶ cells/ml with a nominal multiplicity of infection of 0.5 for each virus (10 ml of each P1 virus stock for a 500 ml culture).

### Protein Purification

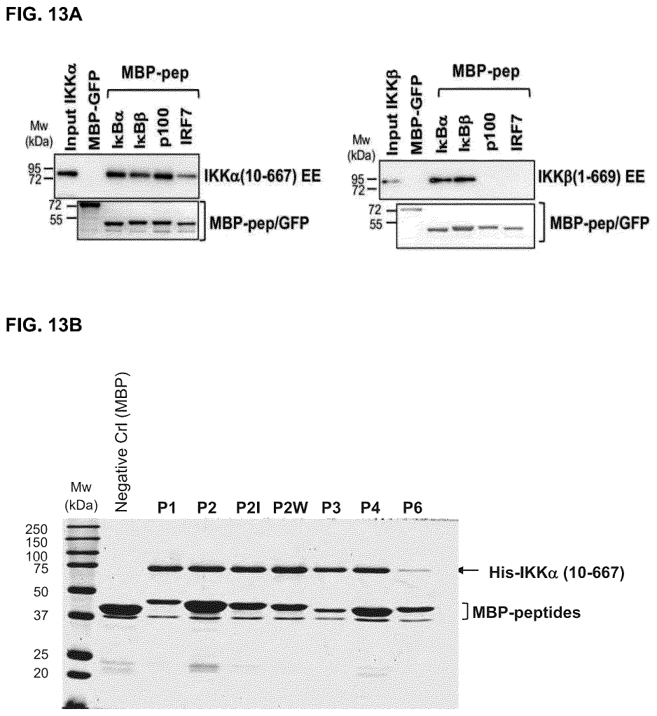

IKKα and IKKβ homodimers. Purification of 6×His-IKKα(10-667) EE and 6×His-IKK(1-669) EE constructs was carried out according to previously published protocols with some modifications according to the type of experiments the samples were used for. Briefly, SF21 culture pellets expressing IKKα or IKKβ proteins were resuspended in lysis buffer (25 mM Tris-HCl pH 8.0, 200 mM NaCl, 10 mM imidazole, 10% glycerol, 2 mM DTT, 2 μg/ml DNaseI, 0.2 μg/ml RNAse, Roche Complete protease inhibitor cocktail) and lysed by mild sonication. Lysates were centrifuged at 14500 g for 45 minutes at 4° C. The supernatants were applied by gravity flow to a plastic column containing pre-equilibrated Ni2+-NTA agarose resin (1.5 ml of dry bead volume per liter of SF21 expression). The resin was extensively washed by four consecutive steps: with equilibration/wash buffer 1 (25 mM Tris-HCl pH 8, 200 mM NaCl, 10 mM imidazole, 10% glycerol, 2 mM DTT), with wash buffer 2 (25 mM Tris-HCl pH 8, 200 mM NaCl, 25 mM imidazole, 10% glycerol, 2 mM DTT), with wash buffer 3 (25 mM Tris-HCl pH 8, 1 M NaCl, 10% glycerol, 2 mM DTT) and with equilibration/wash buffer 1. Hence, IKKα or IKKβ proteins were eluted with 5 dry bead volumes of elution buffer (25 mM Tris-HCl pH 8, 150 mM NaCl, 5% glycerol, 250 mM imidazole, 2 mM DTT). 6×His-constructs purity and stability are very good without contamination, as shown by the SDS-PAGE gel with Coomassie stain in FIGS. 13A-13B.

Next, in the case of IKKα and IKKβ proteins to be used for the in vitro pulldown and kinase activity assays, samples were concentrated to 500 ml and incubated in presence of 1 mM ATP plus additives (20 mM MgCl2, 20 mM b-Glycerophosphate, 10 mM NaF, and 1 mM sodium orthovanadate) for 1 hour on ice. Samples were then purified by gel filtration chromatography using a Superdex 200 10/30 column (GE Healthcare) pre-equilibrated in gel filtration buffer 1 (20 mM Tris-HCl pH 8.0, 150 mM NaCl, 5% Glycerol, 2 mM DTT). In the case of cross-linking mass spectrometry experiments, concentrated IKKβ samples were incubated with 0.2 mM ML120B (Sigma) inhibitor (instead of ATP) plus additives for 1 hour on ice and then loaded on the Superdex 200 10/30 column (GE Healthcare) pre-equilibrated in gel filtration buffer 2 (20 mM HEPES, pH 8.0, 150 mM NaCl, 5% glycerol, and 1 mM TCEP).

For the ITC and crystallization experiments, the 6×His tag was remove from the IKKα and/or IKKβ constructs by proteolytic digestion. For this, after elution from the Ni2+-NTA resin, the sample was placed in a dialysis device (Gebaflex) in presence of home-made TEV enzyme and dialyzed overnight against 2 L of dialysis buffer (20 mM Tris-HCl PH 8, 150 mM NaCl, 2 mM DTT, 5% glycerol). Then, the sample was loaded onto a disposable plastic column filled with 1 ml of Ni2+-NTA resin (dry bead volume) pre-equilibrated in dialysis buffer and the flow-through containing the cleaved IKKβ protein collected. Samples were then concentrated to 2 ml and incubated with ML120B inhibitor at a concentration equivalent to a protein: inhibitor stoichiometric ratio of 1:20 plus additives (see above) for 1 hour on ice. Finally, the sample was applied to a Superdex 200 16/60 column (GE Healthcare) equilibrated in gel filtration buffer 2 (20 mM HEPES pH 8.0, 250 mM NaCl, 2 mM TCEP). Collected fractions were pooled together and the NaCl concentration adjusted to 150 mM. Purified IKKα and IKKβ samples were stored at 4° C. for up to 5 days.

IKKα/β heterodimer. SF21 cells expressing the Strep-IKKα(10-667) EE and 6×His-IKKβ(1-669) EE proteins were resuspended in lysis buffer (100 mM Tris-HCl pH 8.0, 150 mM NaCl, 10% glycerol, 2 mM DTT, 2 μg/ml DNaseI, 0.2 μg/ml RNAse, Roche Complete protease inhibitor cocktail) and lysed by mild sonication. Lysates were centrifuged at 14500 g for 45 minutes at 4° C. The supernatants were applied by gravity flow to Strep-Tactin XT 4F resin (IBA-lifesciences) pre-equilibrated in wash buffer (100 mM Tris-HCl pH 8, 150 mM NaCl, 10% glycerol, 1 mM EDTA, and 2 mM DTT). The resin was extensively washed with wash buffer and the proteins eluted with elution buffer (100 mM Tris-HCl pH 8, 150 mM NaCl, 10% glycerol, 2 mM DTT, and 50 mM biotin). The eluate was then applied to Ni²⁺-NTA resin, which was washed and eluted as described above for the IKK homodimers. IKKα/β heterodimer samples were then concentrated and incubated in presence of 1 mM ATP plus additives (see above) for 1 hour on ice. Finally, samples were purified by gel filtration chromatography using a Superdex 200 10/30 column (GE Healthcare) pre-equilibrated in gel filtration buffer 1 (20 mM Tris-HCl PH 8.0, 150 mM NaCl, 5% Glycerol, 2 mM DTT).

6×His-IκBα. Pellets of 500 ml bacterial cultures expressing 6×His-IκBα proteins for kinase activity studies were resuspended in 50 ml of lysis buffer (25 mM Tris-HCl PH 8, 250 mM NaCl, 10 mM imidazole, 5% glycerol, 0.2% NP40, 2 mM DTT, 0.2 μg/ml DNase I, 0.2 μg/ml RNAse, Roche Complete protease inhibitor cocktail) and lysed by sonication. Lysates were clarified by centrifugation at 96 000 g for 45 minutes at 4° C. The supernatant was applied by gravity flow to plastic column containing 1 ml (dry bead volume) of pre-equilibrated Ni2+-NTA agarose resin. The resin was extensively washed in four steps: first with equilibration/wash buffer 1 (25 mM Tris-HCl PH 8, 200 mM NaCl, 10 mM imidazole, 2 mM DTT), then with wash buffer 2 (25 mM Tris-HCl PH 8, 200 mM NaCl, 25 mM imidazole, 2 mM DTT), wash buffer 3 (25 mM Tris-HCl pH 8, 1 M NaCl, 2 mM DTT) and finally with equilibration/wash buffer 1. Subsequently, 6×His-IκBα proteins were eluted with 2.5 ml of elution buffer (25 mM Tris-HCl PH 8, 200 mM NaCl, 250 mM imidazole, 2 mM DTT). Finally, imidazole was eliminated using a PD-10 desalting column (GE Healthcare) equilibrated in 20 mM Tris-HCl, 150 mM NaCl, 2 mM DTT. Samples were concentrated, flash frozen in liquid nitrogen and stored at −80° C.

MBP-fusions. MBP-IκBα and MBP-peptide constructs were coupled to the amylose resin for the pulldown experiments. Pellets of 10-25 ml of bacterial culture were resuspended in 2.5 ml of lysis buffer (20 mM Tris-HCl pH 8, 250 mM NaCl, 10% glycerol, 0.2% NP40, 2 mM DTT, 0.2 μg/ml DNase 1, 0.2 μg/ml RNAse, Roche Complete protease inhibitor cocktail) and lysed by sonication. Lysates were centrifugated using a benchtop centrifuge at maximal speed and at 4° C. Clarified lysates were incubated with 100 ml (dry bead volume) of previously equilibrated amylose resin (New England Biolabs) for 45 minutes at 4° C. on rotating wheel. Resins were washed first with equilibration/wash buffer 1 (20 mM Tris-HCl pH 8, 250 mM NaCl and 2 mM DTT) and then with wash buffer 2 (20 mM Tris-HCl pH 8, 150 mM NaCl and 2 mM DTT). Hence, resins coupled to MBP-fusion constructs were stored at 4° C. up to one week. MBP-peptide purity and stability are very good without contamination, as shown by the SDS-PAGE gel with Coomassie stain in FIG. 13B.

### MBP-Pulldown

Experiments were performed in PD buffer (20 mM Tris pH 8.0, 150 mM NaCl, 2 mM DTT). For each experiment, 10 μl of amylose resin (dry bead volume) coupled to MBP-fusion constructs were incubated with 500 ml of purified 6×His-IKKα(10-667) EE and 6×His-IKKβ (1-669) EE constructs adjusted at a concentration of 0.3 μM for 2 hours at 4° C. After two quick washing steps with PD buffer, complexes were eluted by incubation with 20 μl of the same buffer supplemented with 20 mM maltose for 15 minutes at 4° C. After mild centrifugation, supernatants were recovered and migrated onto two separate 10% SDS-PAGE gels. One gel was used for Western blot analysis to detect His-IKKα or His-IKKβ using and anti-6×His antibody (Sigma, #SAB4301134), the other for Coomassie staining to detect MBP-fusion proteins. Quantification of band intensity was done using the Image J software.

### GPCA

HEK293T cells were transfected using the reverse transfection method. Transfection mixes containing 100 ng of pSPICA-N2 and 100 ng of pSPICA-N1 plasmids expressing test proteins plus JetPEI® (Polyplus transfection) were dispensed in white 96-well plates. HEK293T cells were then seeded on the DNA mixes at a concentration of 4.2×104 cells per well. 48 hours after transfection, cells were washed with 50 μl of PBS and lysed with 40 μl of Renilla lysis buffer (Promega, E2820) for 30 minutes with agitation. Gaussia princeps luciferase enzymatic activity was measured using a Berthold Centro LB960 luminometer by injecting 50 μl per well of luciferase substrate reagent (Promega, E2820) and counting luminescence for 10 seconds. Results are expressed as a fold change normalized over the sum of controls, specified herein as normalized luminescence ratio (NLR) 29. For a given protein pair A/B, NLR=(Gluc1-A+Gluc2-B)/[(Gluc1-A+Gluc2)+(Gluc1+Gluc2-B)]. All interactions were measured in three independent experiments, with each experiment being carried out in triplicate. The expression levels of Gluc1- and Gluc2-fused proteins were checked by Western blotting. 24 hours post-transfection, HEK293T cells were collected, lysed by incubation on ice and centrifuged. Aliquots of cleared supernatants were loaded onto an 10% SDS-PAGE gel. Proteins were detected using a polyclonal anti-Gluc antibody, which allows to detect both Gluc1 and Gluc2 fragments, albeit with lower efficiency for the Gluc2 fragment.

### ITC

All experiments were performed in ITC buffer (20 mM HEPES pH 8.0, 150 mM NaCl, 1 mM TCEP) unless otherwise stated. Purified IKKα(10-667) EE and IKKβ(1-669) EE protein samples were dialyzed overnight against ITC buffer at 4° C. Just before the ITC experiments, IKKα or IKKβ samples were concentrated, incubated at room temperature for 30 minutes and centrifuged at 11000 g to remove eventual precipitate. The final concentrations of IKKα and IKKβ in the sample cell varied between 22 and 50 μM depending on the measurement. HPLC purified synthetic peptides (IκBα pep) comprising residues 297-317 of IκBα were further desalted using a Nap5 (GE Healthcare, #17-0853-01) column equilibrated in milliQ water, lyophilized and then resuspended in ITC buffer. The concentration of peptides in the syringe varied between 220 and 975 μM depending on the measurement.

All ITC measurements were performed using a PEAQ-ITC MALVERN instrument. The titration parameters were set as following: temperature 25° C., reference power 10 μcal/s, feedback=high, stirring speed 750 RPM, initial delay 60 s, first injection 0.4 μl in 0.8 s and the remaining 19 injections were 2 μL in 4 s with 120 s of spacing. The data were fitted to a 1:1 binding model. All data were processed using NITPIC software and global fitting of two datasets was carried out using SEDPHAD.

### In Vitro Kinase Assay

Using recombinant IKKβ. Reactions were performed in kinase buffer (50 mM Tris-HCl at pH 8.0, 100 mM NaCl, 10 mM MgCl2, 2 mM MnCl2, 0.5 mg/mL BSA, 20 mM β-glycerol phosphate, 400 μM Na2VO4, 2 mM DTT, and 400 μM ATP) using 0.5 nM of purified 6×HisIKKβ(1-669) EE protein and 1 μM of purified full-length 6×His-IκBα wt or mutant constructs. Reaction mixtures were set-up in a total volume of 70 μl and incubated at 30° C. During the course of the incubation, 15 μl aliquots were sampled at the indicated times (0, 5, 10 and 30 min) and the reactions stopped by addition of Laemmli sample buffer plus heating for 5 minutes at 95° C. The products were analysed on a 10% SDS-PAGE gel and Western blotting using an antibody that specifically recognizes phosphorylated Ser32/Ser36 residues of IκBα (antiphospho IκBα, Cell Signaling Technology, #9246) and anti-6×His (Sigma, #SAB4301134) antibodies. The relative amounts of phosphorylated IκBα were quantified using ImageJ.

Using endogenous IKK complex. IKK was immunoprecipitated from HEK293T cells. 3 petri dishes (100×15 mm, corning) of cells at 70% confluence were induced with 10 nM recombinant TNFα (abcam, #ab9642) for 10 minutes, and then washed twice by cold PBS. The whole-cell extracts were prepared by addition of 1 ml of lysis buffer (50 mM HEPES pH 8, 200 mM NaCl, 0.5% NP-40, 1 mM EDTA, 10 mM KCl, 1 mM DTT, Roche cOmplete™ protease inhibitors cocktail, 50 mM NaF, 50 mM b-glycerophosphate and 1 mM Na orthovanadate) and homogenized by passing through a 21-gauge needle for 5-10 times. After 30 minutes of incubation on ice, the extracts were centrifuged at 11,000 g and at 4° C. The cleared supernatants were incubated with 15 μl of anti-NEMO antibody (Santa Cruz biotech, #F-10) overnight at 4° C. and, then, immunoprecipitated with 150 μl of protein G Sepharose beads (GE, #17-0618-01) for 2 hours at 4° C. Beads were washed with wash buffer (50 mM Tris, pH 8, 150 mM NaCl, 5 mM b-glycerophosphate, 0.1 mM Na orthovanadate, 10 mM MgCl2, 2 mM DTT) and then resuspended with was buffer to a final volume of 150 μl.

Kinase activity reactions were performed in kinase buffer (defined above) using 50 μl immunoprecipitant and 1 μM of purified full-length 6×His-IκBα wt or mutant constructs. In the case of experiments aimed at evaluating inhibition of IKKβ activity by peptides comprising the docking motif, the reaction mixtures were additionally supplemented with 500 μM of synthetic peptides (IκBα pep) comprising residues 297-317 of IκBα. Reaction mixtures were set-up in a total volume of 150 μl and incubated at 30° C. During the course of the incubation, 30 μl aliquots were sampled at the indicated times (0, 5, 10 and 30 minutes) and the reactions stopped by addition of Laemmli sample buffer plus heating for 5 minutes at 95° C. The reaction products were analysed on a 10% SDS-PAGE gel and Western blotting using anti-phospho-IκBα (Cell Signaling Technology, #9246), anti-IKKβ (cell signaling, #2684s) and anti-6×His (sigma, #SAB4301134) antibodies. The relative amounts of phosphorylated IκBα were quantified using ImageJ.

### In Cellulo Inhibition of NF-κB Signaling

The mRNAs coding for the mScarlet-E3-IκBα pep fusions were transcribed from the T7 promoter using HiScribe™ T7 ARCA mRNA Kit (BioLabs, E2060S) according to the manufacturer recommendations. 0.5 μg mRNAs were electroporated in 1·10⁵ MRC-5 cells using Neon™ transfection system (Invitrogen, MPK1096). 24 hours after electroporation, cells were treated or not with TNFα (R&D system, 210-AT) at 20 ng/ml for 30 minutes. Hence, cells were fixed with 4% (w/v) paraformaldehyde for 30 minutes and then permeabilized with 0.2% Triton X-100 for 5 minutes in PBS buffer, 10% FCS. Cells were incubated with anti-p65 antibody (1/2000 dilution) (Santa-Cruz biotechnology, sc-800) followed by an anti-mouse mAb-Alexa-Fluor 488 conjugated (Life Technology, A11001). Coverslips were then mounted with Fluoromount G containing 4′, 6′-diamidino-2-phenyleindole (SouthernBiotech, 0100-20). Treated cells were analyzed by fluorescence microscopy (Leica DM5500B and LAS AF software). Image processing was performed using ImageJ 2.3.0. Data analysis was performed using Prism 9 software. Statistical analysis was performed by the ANOVA t test.

x-Ray Crystallography

Crystallization. The purified IKKβ(1-669) EE construct in 20 mM HEPES pH 8, 150 mM NaCl, 2 mM TCEP was concentrated to 15 mg/ml, mixed with a 1.5 molar excess of synthetic wt IκBα pep and incubated on ice for 1 hour. Crystallization experiments were performed by the sitting drop vapour diffusion method at 277 K using a Mosquito Crystal nanolitre dispensing robot (SPT Labtech) and several commercially available screens (The Berkeley screen, the JCSG+ suite, the ProComplex suite, the Classics suite, the PEGs suite, and the Wizard Classic 1 & 2 suite). 0.1 μl of the sample solution was mixed with 0.1 μl of the reservoir solution and equilibrated against 50 μl of reservoir solution in 96-well MRC2 sitting drop vapour diffusion crystallisation plates (Molecular Dimensions). The plates were stored at 277 K and automatically imaged using a RockImager 1000 (Formulatrix).

Small initial crystals (~50×15×15 μm) appeared after 2 days in condition 4 of the Berkeley screen (1.8 M (NH4)2SO4, 0.1 M Bis-Tris HCl pH 6.5, 10% 2-propanol) but appeared multiple. After manual reproduction of the condition, large single crystals (~500×80×40 μm) grew after 8 days. The crystals were transferred to 2 M Li2SO4, 0.1 M Bis-Tris HCl pH 6.5 before flash cooling in liquid nitrogen.

Data collection and processing, and structure determination. X-ray diffraction data were collected at 100 K on an EIGER X 9M detector (Dectris) at the Proxima 2A beamline of Synchrotron SOLEIL (Paris). 360° of data were collected using 0.1° rotation and 0.025s exposure per image at 100% beam intensity at an energy of 12.65 keV (wavelength=0.98 Å). The data were processed and scaled using XDS (Kabsch, 2010) before anisotropic truncation and correction was performed using the STARANISO server (Tickle et al., 2018). The crystal diffracted anisotropically to 4.2 Å (6.8 Å in the worst direction) and belonged to the C-centered monoclinic space group C2 with unit cell dimensions a=226.3 Å, b=136.8 Å, c=204.4 Å, β=91.45°.

The structure was solved by molecular replacement using PHASER in the PHENIX suite using a monomer extracted from the structure of the asymmetric dimer of human IκB kinase β as a search model. The asymmetric unit contains five copies of the protein (two dimers, and one monomer that forms a dimer with itself over a crystallographic 2-fold rotation axis), with a corresponding Matthews' coefficient (Matthews, 1968) of 4.1 Å3 Da-1 and a solvent content of 69.9%. Refinement of the structures was performed using PHENIX and BUSTER followed by with iterative model building in COOT.

The refined IKKβ model and structure factor amplitudes have been deposited in the PDB under accession number 80 MV.

### Cross-Linking Mass Spectrometry (CLMS)

Sample preparation. Purified 6×His-IKKβ(1-699) EE protein in 20 mM Hepes, pH 8.0, 150 mM NaCl, 5% glycerol and 1 mM TCEP was adjusted at a concentration of 12 μM and incubated with 10-fold molar excess of C308L/R314K IκBα pep on ice for 2 hours in presence of 10 mM MgCl2. For crosslinking with EDC/Sulfo-NHS, the IKKβ/peptide mixture was incubated with 5 mM EDC (Thermo Fisher Scientific, #A35391) and 10 mM Sulfo-NHS (Thermo Fisher Scientific, #A39269) at RT for 30 minutes in activation buffer (100 mM MES, pH 5.5, 500 mM NaCl, 10 mM MgCl2, 2.5% glycerol and 1 mM TCEP) in a total volume of 100 μL. Then, an equal volume of 10′ PBS was added to increase the pH of the sample, which was further incubated at RT for 2 hours. To quench the crosslinking reaction hydroxylamine (Thermo Fisher Scientific, #26103) was added to a final concentration of 10 mM. For crosslinking with BS3, the IKKβ/peptide mixture was incubated with 600 μM BS3 at 4° C. for 2 hours in a total volume of 100 μL, then the reaction was quenched by incubation with 50 mM fresh ammonium bicarbonate at RT for 30 minutes. For crosslinking with Sulfo-SDA, the IKKβ/peptide mixture was incubated with different concentrations (0.2, 0.5, 1, and 2 mM) of Sulfo-SDA (Thermo Fisher Scientific, #26173) at 4° C. for 2 hours in the dark. Then, the mixture was photoactivated by UV irradiation for 15 or 30 minutes. The UV lamp (set at 6 watts, 365 nm) was placed at 2 cm from the sample. Finally, the reaction was quenched by incubation with 50 mM fresh ammonium bicarbonate at RT for 30 minutes.

Crosslinked samples were separated using an 8% SDS-PAGE that was stained using Quick Coomassie (Neo Biotech, #NB-45-00078). IKKβ-peptide bands were cut into about 1 mm′1 mm cubes, dried by acetonitrile, and stored at −20° C. until analysis. Proteins in the gel bands were reduced and cysteines were modified with iodoacetamide. Digestion with trypsin (Thermo Scientific Pierce) was occurred over night at 37° C. The resulting tryptic peptides were extracted and desalted using C18 StageTips. Samples were eluted just prior to analysis by LC-MS/MS.

LC-MS analysis. LC-MS/MS analysis was performed on an Orbitrap Fusion Lumos Tribrid mass spectrometer (Thermo Fisher) coupled on-line with an Ultimate 3000 RSLCnano system (Dionex, Thermo Fisher). Samples were separated on a 50-cm EASY-Spray column (Thermo Fisher). Mobile phase A consisted of 0.1% (v/v) formic acid and mobile phase B of 80% (v/v) acetonitrile with 0.1% (v/v) formic acid. Flow rates were 0.3 μl/minute using a gradient from 2% to 45% mobile phase B over 90 minutes. Mass spec data were acquired in data-dependent mode using the top-speed setting with a three second cycle time. For every cycle, the full scan mass spectrum was recorded using the Orbitrap at a resolution of 120,000 in the range of 400 to 1,600 m/z. Ions with a precursor charge state between 3+ and 7+ were isolated and fragmented. Each precursor was fragmentated by higher-energy Collisional Dissociation (HCD) at 26%, 28% and 30%. The fragmentation spectra were then recorded in the Orbitrap with a resolution of 60,000. Dynamic exclusion was enabled with single repeat count and 60-seconds exclusion duration.

Data analysis. A recalibration of the precursor m/z was conducted based on high-confidence (30% sequence coverage on each peptide and a delta score that was >30% of the score, for all crosslinkers. Additionally for SDA, there had to be at least one crosslinker containing fragment for each peptide. This filtering was done with xiFDR v.2.2.beta5 (PMID: 31556486). No decoys passed these thresholds for any of the datasets. All raw and processed data are available on JPOST (PMID: 27899654) and ProteomeXchange with identifier PXD037534

### Protein-Peptide Docking Calculations

The IKKβ/IκBα pep complex model was calculated using the HADDOCK2.4 webserver. The IKKβ AB homodimer was used as a starting structure, whereas IκBα pep (residues 297-317 of the wt IκBα sequence) was generated in an extended conformation. The IκBα pep was docked to one of face of the symmetric IKKβ homodimer. IKKβ interface residues binding to IκBα pep were first identified from the quantitative analysis of CLMS data using the DisVis tool. These residues were maintained as active throughout the docking protocol. Thirty-nine unique distance restrains derived from CLMS data were imposed as ambiguous restraints (with OR statement), so that they could map to residues from either chain of the IKKβ homodimer. Restraints derived from links obtained with sulfo-SDA spanned 2.5-22.5 Å (Cb-Cb), whereas EDC/NHS crosslinks were set to 1.5-7 Å (Cb-Cb). The number of molecular dynamics steps in the rigid body and flexible refinement stages was increased to allow for sampling of larger conformational changes. In particular, the steps for rigid body high temperature torsion angle dynamics, first rigid body cooling, second cooling stage and fully flexible interface third cooling stage were all increased to 2000. 1000 structures were generated in it0 and the top 200 structures were selected for refinement and clustered. The entire peptide was set to be fully flexible. Docking solutions were clustered on the basis of interface r.m.s.d. with a cutoff of 5 Å. The best scoring structure from the best scoring cluster is taken as solution.

### Results

IKKα and IKKβ Dock to a YDDφXφ Motif within IκBα

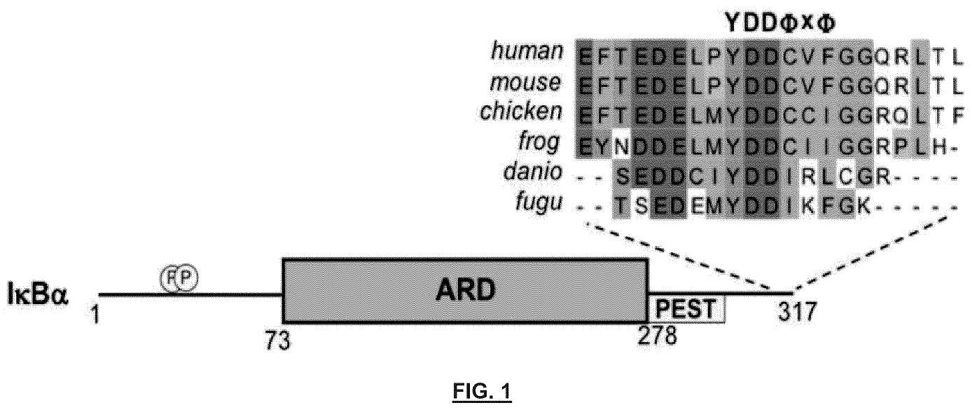

The canonical IκBα substrate comprises a disordered N-terminal region (residues 1-72), which hosts phosphor-Ser32 and phospho-Ser36 residues within the DpSGΨXpS/T substrate motif targeted by IKKβ, an ankyrin repeat domain (residues 73-278, ARD) and a disordered C-terminal region containing a PEST sequence (residues 278-317) (FIG. 1). Previous work by Hu and coworkers suggested the existence of a binding site for IKKβ located somewhere within the ankyrin repeat domain and C-terminal regions of IκBα. To identify this site, in vitro pulldown experiments employing recombinant purified proteins were performed. For this, the inventors produced IκBα constructs fused to the C-terminus of the maltose binding protein (MBP) and a nearly full-length (residues 1-669) and constitutively active (S177E/S181E) mutant construct of human IKKβ (named IKKβ(1-669) EE hereafter), which is very similar to the previously crystallized fragment of this protein. The IKKβ homodimer was purified by affinity and size exclusion chromatography. The interaction of the IKKβ homodimer with full-length IκBα becomes clearly visible only at relatively high concentrations of IKKβ. Surprisingly, deletion of the N-terminal region of IκBα enhances binding to IKKβ. In contrast, further deletion of the C-terminal region abolishes the interaction. A 17 amino acid peptide from the IκBα C-terminus (residues 301-317, named IκBα pep(s) hereafter) is instead necessary and sufficient to interact with IKKβ at levels comparable with those of the N-terminal truncated construct. Serine mutagenesis of IκBα pep(s) indicates that Tyr305, Asp306, Asp307, Cys308 and Phe310 residues are essential for the interaction with IKKβ. Consistently, sequence alignments reveal that these residues are conserved in IκBα proteins from different species and are part of a motif with a Y₁D₂D₃φ₄X₅φ₆ consensus (where Y is a tyrosine, D is an aspartic acid, φ is a hydrophobic amino acid and X is any amino acid) (FIG. 1). This motif appears to be flanked on the N-terminal side by a patch of acidic residues (residues 297-302), which are conserved in several species.

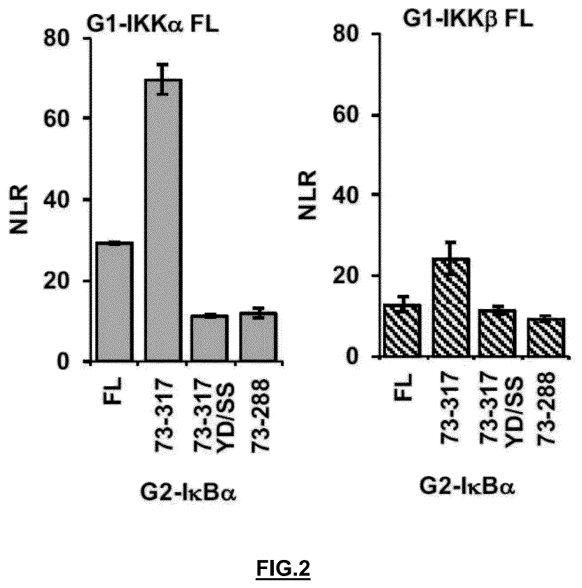

Next, IKKα and IKKβ interactions with IκBα in cellulo were analyzed using the Gaussia princeps protein complementation assay (GPCA). Here, full-length IKKα or IKKβ proteins and IκBα constructs are fused to C-terminal extremities of the G1 and G2 fragments of the luciferase, respectively. Pairwise combinations of G1-IKKα/β and G2-IκBα are co-transfected in HEK293T cells and the interactions monitored following the activity of the reconstituted luciferase. Interestingly, both IKKα and IKKβ interact with full-length IκBα, with IKKα showing higher binding responses (FIG. 2). Truncation of the N-terminal region of IκBα again appears to enhance binding to both IKKα and IKKβ (FIG. 2, compare IκBα FL with IκBα (73-317)), an effect which may be related to the absence, in these assays, of NF-κB dimers relieving steric hindrance contributions of this region. Instead, further deletion of the disordered C-terminal region or the double serine substitution at positions Y₁ and D₃ of the motif (i.e. Y305S/D3087S, named YD/SS hereafter) strongly decrease the interactions with both IKKs (FIG. 2, compare IκBα(73-317) with IκBα(73-288) and IκBα(73-317) YD/SS).

The inventors then proceeded to reconstitute the IKKα/β heterodimer by co-expression of 6×His-IKKβ(1-669) EE and Strep-IKKα(10-667) EE constructs in insect cells and purification by two consecutive affinity-chromatography steps (Ni²⁺-NTA and Strep-Tactin) plus gel filtration (see methods section). Results from MBP-pulldown analyses show that the IKKα/β heterodimer efficiently binds to IκBα pep.

To evaluate binding affinities, isothermal titration calorimetry (ITC) experiments were performed using a synthetic peptide that comprises the YDDφXφ motif plus the N-terminal acidic patch (residues 297-317, named IκBα pep hereafter) and purified IKKβ(1-669) EE homodimer or an equivalent construct of the IKKα homodimer (i.e. IKKα(10-667) S176E/S180E, named IKKα(10-667) EE hereafter). Results show higher binding affinity for the interaction with IKKα (K_{D}=9 μM) as compared to IKKβ (K_{D}=40 μM) (see table 1 hereafter). ITC analyses on the IKKα/β heterodimer were prevented by strong aggregation propensity at the concentrations required for these experiments.

TABLE 2
Thermodynamic parameters of IKKα or IKKβ binding
to YDDΦXΦ peptides

| | | K_{D} | ΔH | TΔS | ΔG |
|---|---|---|---|---|---|
| interaction | N | (μM) | (kcal/mol) | (kcal/mol) | (kcal/mol) |
| IKKα/IkBα pep wt | 0.62 | 9.0 | −15.2 | −8.35 | −6.88 |
| IKKα/IkBα pep | 0.65 | 0.35 | −13.5 | −4.71 | −8.80 |
| C308L | | | | | |
| IKKβ/IkBα pep wt | 0.58 | 40 | −15.8 | −9.78 | −6.00 |
| IKKβ/IkBα pep | 0.63 | 2.6 | −12.2 | −4.59 | −7.61 |
| C308L | | | | | |
| IKKα/p100 pep | 0.60 | 11 | −12.3 | −5.56 | −6.77 |
| IKKα/IRF7 pep | 0.6 | 90 | −6.7 | −1.2 | −5.5 |

All ITC experiments were performed at 25° C. The data were fitted to a 1:1 binding model. N refers to the binding stoichiometry of IKKα/β: pep, meaning that approximately half of total IKKα/β concentration binds pep ligand in agreement with the dimer form of IKKα/β being binding competent. The values for the IKKα interaction with IRF7 pep should be regarded as estimates due to low affinity and weakly exothermic binding, and therefore high experimental error and fitting uncertainty. All data were processed using NITPIC software and global fitting of two datasets was carried out using SEDPHAD.

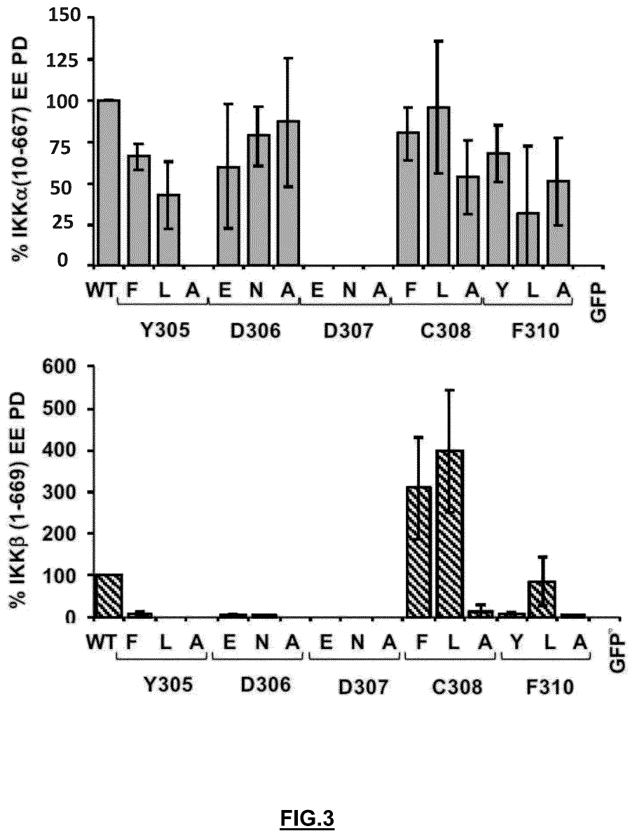

Finally, single amino acid substitutions were introduced at the conserved positions of the motif of IκBα pep(s) (i.e. Y₁, D₂, D₃, φ₄ and φ₆) and the impact on the interactions with IKKβ(1-669) EE or IKKα(10-667) EE was evaluated by pulldown. Most of the mutations tested have a negative impact on the interactions with IKKβ. In contrast, IKKα appears to tolerate a higher number of substitutions, particularly at positions Y₁, D₂ and φ₆ (FIG. 3). Remarkably, two substitutions at the φ₄ position, i.e. C308L and C308F, clearly enhance binding to IKKβ (FIG. 3). Subsequent ITC analyses showed that the C308L substitution enhances IκBα pep affinities to both IKKα and IKKβ, by 25- and 15-fold, respectively (K_{D}=350 nM for IKKα and K_{D}=2.6 UM for IKKβ).

Together, these results demonstrate that homo- and heterodimers of the catalytic IKKα and IKKβ subunits dock to a YDDφXφ motif located in the C-terminal region of the IκBα substrate. IKKα appears to interact with this motif with higher affinity as compared to IKKβ. In addition, sequence variations at the conserved positions of the motif enable modulation of the interaction affinities in both positive and negative ways.

The Affinity of the Docking Interaction Correlates with IKK Activity

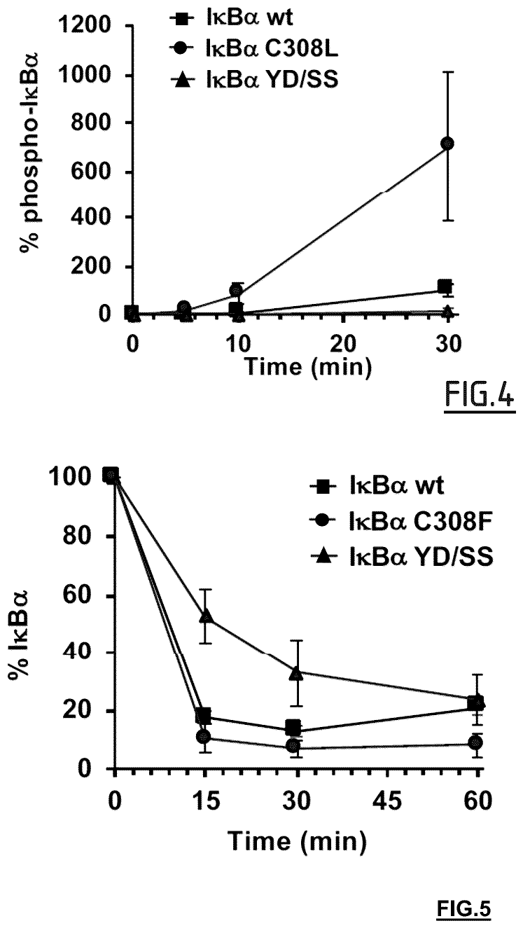

In the next set of experiments, the inventors assessed the impact of the docking interaction on IKK activity. To this end, in vitro kinase assays were performed using purified full-length wt or mutant IκBα proteins and endogenous canonical IKK, which was immunoprecipitated from HEK293T cells using an anti-NEMO antibody. Results show that Ser32/Ser36 phosphorylation is highest for the IκBα C308L mutant with improved docking affinity, whereas IκBα YD/SS is not phosphorylated under these assay conditions (FIG. 4). Similar results were obtained by an experiment that uses recombinant purified IKKβ(1-669) EE instead of endogenous canonical complex.

Then, the degradation profiles of wt or mutant IκBα proteins upon activation of the canonical NF-κB pathway were evaluated. In these experiments, IκBα constructs are retro-transduced in mouse embryonic fibroblast (MEF) lacking the IκB family genes (MEF IκBα/IκBβ/IκBε KO) and their protein levels monitored at selected time points after TNFα stimulation. As expected, the S23A/S36A (SS/AA) mutation at the phospho-acceptor residues completely suppresses IκBα degradation. Mutations at the YDDφXφ docking motif have milder yet significant effects on IκBα levels. In particular, degradation of IκBα YD/SS is clearly slower as compared to that of wt IκBα (FIG. 5). In addition, a small enhancement of degradation relative to the wt protein is also observed for the higher affinity IκBα C308F mutant (FIG. 5), which was used here due to the low expression levels of IκBα C308L in MEFs.

Taken together, these results indicate that the affinity of the docking interaction correlates with both phosphorylation and degradation of the IκBα substrate. Yet, the residual degradation of the IκBα YD/SS mutant suggest that additional determinants contribute to substrate recruitment, which likely involve the substrate motif and adjacent Asp27 and Asp28 residues that were previously reported to interact with the NEMO subunit.

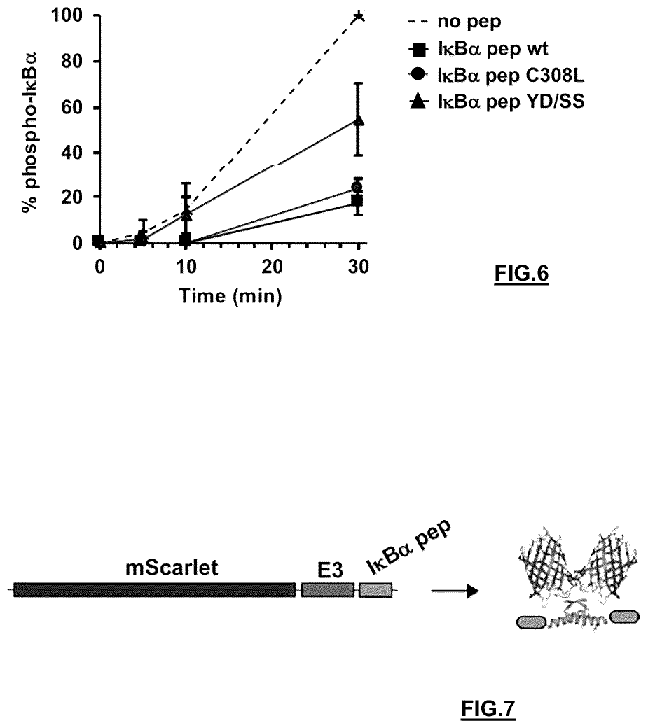

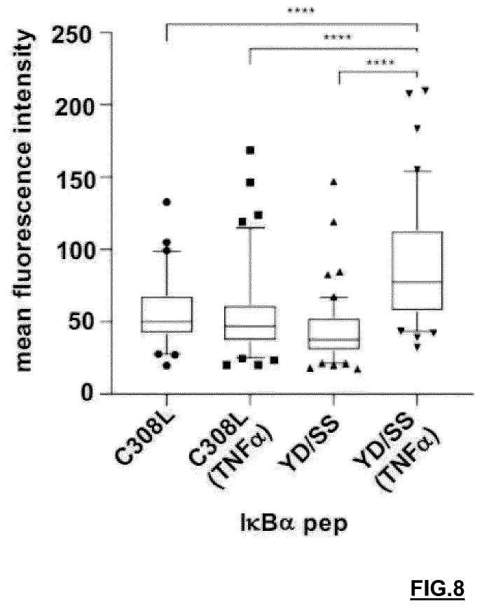

The MBP-IκBα pep fusion comprising the C308L mutation is able to capture endogenous IKKβ from clarified extracts of HEK293T cells, raising the possibility that docking motif peptides could act as competitive inhibitors of IKK. Consistently, results from in vitro kinase experiments performed in presence of synthetic IκBα pep variants indicate that wt and C308L peptides inhibit IκBα phosphorylation more efficiently than the YD/SS negative control peptide (FIG. 6). To evaluate the inhibitory activity of these peptides in vivo, bivalent fusion constructs comprising (from N- to C-terminus) the mScarlet protein for detection by immunofluorescence, the E3 peptide that promotes dimerization to enhance binding through avidity contributions and the IκBα pep YD/SS or C308L sequences were constructed (FIG. 7). These constructs were transfected as mRNAs into Human Fetal Lung Fibroblast (MRC-5) cells and activation of NF-κB signaling monitored by following nuclear translocation of p65. As expected, in unstimulated conditions p65 is found in the cytoplasm. Upon TNFα induction, p65 translocates into the nucleus only in the case of the IκBα pep YD/SS negative control, whereas IκBα pep C308L mutation efficiently retains p65 in the cytoplasm like in the unstimulated conditions (FIG. 8). These results provide evidence that peptides incorporating the YDDφXφ docking motif are sufficient to block NF-κB signaling.

### Structural Studies of the IKKβ/IκBα Pep Complex

Several crystallization screenings were carried out on samples of IKKα or IKKβ homodimers bound to synthetic IκBα pep wt or C308L. Most of the crystals obtained displayed poor x-ray diffraction patterns, reflecting previously reported difficulties linked to high resolution structural studies on these dimers. However, the inventors were able to identify one crystal grown from a sample of IKKβ(1-669) EE mixed to wt IκBα pep, which diffracted synchrotron radiation to a resolution of 4.15-6.8 Å (anisotropic diffraction), allowing for structure determination by molecular replacement. The asymmetric unit of the crystal contains five IKKβ protomers. Four protomers form two distinct homodimers (A-B and C-D) with the characteristic “scissor-like” structure mediated by contacts involving the C-terminal portions of the SDD domains (dimerization region). The fifth protomer of the unit cell homodimerizes with an IKKβ molecule from a neighboring unit cell. The conformations of the A-B and C-D homodimers differ in the extent of the splaying apart of the KD-ULD portions, with the A-B homodimer displaying a relatively closed conformation compared to the C-D homodimer. B-factor analysis indicates lower flexibility for the helices of the SDD domain in the A-B homodimer than in the C-D homodimer. Whereas data recapitulate previously observed structural features of IKKβ, the inventors were unable to assign electron density to the IκBα pep due to the lack of high resolution. The inventors therefore turned to cross-linking mass spectrometry (CLMS) to independently map the IκBα binding site on IKKβ. For this, purified IKKβ (1-669) EE was mixed to a 10-fold molar excess of synthetic IκBα pep C308L/R314K, which incorporates an additional Arg to Lys substitution to improve peptide cross-linking efficiency. Protein samples were cross-linked using three distinct agents with different chemistries and spacer lengths: EDC/sulfo-NHS, sulfo-SDA and BS3. Protein-peptide cross-linked species were isolated by migration on SDS-PAGE and analysed by MS. Despite the large excess of peptide added, the totality of the intermolecular IKKβ-peptide cross-links identified in the different samples map to the SDD domain of IKKβ, strongly supporting the specificity of this interaction. EDC/sulfo-NHS, a zero-length cross-linker catalysing amide bond formation, provided 10 cross-links between Glu or Asp residues of the N-terminal acidic patch of IκBα pep and Lys residues of the dimerization region of IKKβ. Consistently, the long-range BS3 cross-linker identified 5 amine-to-amine links between the peptide's N-terminus and the same Lys residues, thereby confirming the proximity of the N-terminal acidic patch of IκBα pep to the IKKβ dimerization region. Sulfo-SDA, a short spacer (3.9 Å) cross-linker that conjugates primary amines or hydroxyls with any amino acid group after UV photoactivation, provided 26 links. Many of these links involve IKKβ residues Met468 and Lys469, which are located in the middle portion of the SDD domain, between the dimerization region and the region making contacts to the KD and ULD domains. Consistently, a small lobe of extra electron density can be seen in proximity of these residues in the A-B homodimer of the crystal unit cell, suggesting that IκBα pep might be bound at this dimer but not entirely visible at this resolution. Quantitative analysis of the ensemble of the CLMS data using the DisVis tool of the HADDOCK webserver indicates that the volume accessible to IκBα pep for interaction with IKKβ is consistent with a binding interface that corresponds to the SDD dimerization and middle regions of the IKKβ homodimer. These results are in agreement with findings from Hu and coworkers showing that constructs of the isolated SDD domain of IKKβ are sufficient for pulldown of the IκBα substrate.

In the next step, IκBα pep was docked to the IKKβ A-B homodimer crystal structure using CLMS-derived distance restraints and the HADDOCK software. Eight acceptable clusters were obtained based on the HADDOCK score and interface root-mean-square deviation (RMSD). In all clusters, IκBα pep binds in an extended conformation to the two adjacent SDD domains of the IKKβ homodimer, with the YDDφXφ motif accommodated in a groove contributed by helices a 2s and a4s from protomer A, and α2s from protomer B of the SDD middle portions. Structural alignment of the models shows high precision for most of the clusters, with mean within-cluster interface r.m.s.d values ranging between 1.3 and 1.8 Å. The best model from the cluster with the lowest Haddock score (cluster 1) is chosen as a representative model. CLMS distance restraints are in very good agreement with this final model. Most of the over-length cross links involve the N-terminal acidic patch of IκBα pep, suggesting that the peptide may bind to the SDD dimerization region with multiple conformations.

The YDDφXφ binding groove is dominated by polar and positively charged residues, with few hydrophobic contribution observed in the proximity of the Y₁ (Tyr305), φ₄, (Cys308) and φ₆ (Phe310) positions of the motif. Specifically, whereas Tyr305 makes stacking interactions with Met475 of IKKβ, Cys308 and Phe310 are inserted in a cavity contributed by Lys469, Ala473, Val534 and Met538 of the two adjacent SDD domains. The acidic residues of the motif (Asp306 and Asp307) are instead stabilized by a positively charged patch that involves several lysine residues of IKKβ (Lys467, Lys480 and Lys531). Several residues lining the YDDφXφ groove are conserved across species in both IKKα and IKKβ. By contrast, the N-terminal acidic patch of IκBα pep is stabilized by a positively charged surface of the dimerization region of IKKβ that consists of overall less conserved residues (Lys641, Arg645 and Lys652).

The structural model was validated by mutagenesis of IKKβ coupled to binding studies. Specifically, IKKβ mutants were evaluated for interaction with the nearly full-length IκBα (73-317) construct by GPCA or with IκBα pep by MBP-pulldown using recombinant purified IKKβ mutants. Results show that the L654D/W655D mutation in the C-terminal portion of the SDD domain, previously reported to suppress IKKβ dimerization, completely abolishes binding to IκBα in both assays, thereby showing that an intact dimer is required for the interaction with the substrate. Consistently, this same mutation has also been shown to markedly reduce IKKβ kinetic activity. In addition, the inventors also identified single amino acid substitutions introducing charge reversal (K469E and K480E) or affecting hydrophobicity (M475A) at specific positions of the YDDφXφ binding groove, which significantly attenuate the binding response to IκBα. The combination of these substitutions in single constructs (K469E/K480E and K469E/M475A) leads to a stronger reduction of binding in both assays. Significant effects were also observed for charge reversal mutations in the C-terminal helix contacting the N-terminal region of IκBα pep (K641E, R645E, K659E and K664E). In contrast, the R460E mutation appears to significantly reduce binding to the N-terminal truncated IκBα (73-317) construct but not to IκBα pep(s), suggesting that R460 may contribute to interactions involving IκBα residues outside the YDDφXφ motif.

Together the results from the biochemical and structural analyses show that the YDDφXφ motif interacts with a conserved groove at the interface between the two SDD domains of the IKKβ homodimer.

The YDDφXφ Motif Mediates IKKα and IKKβ Interactions with NF-κB Substrates

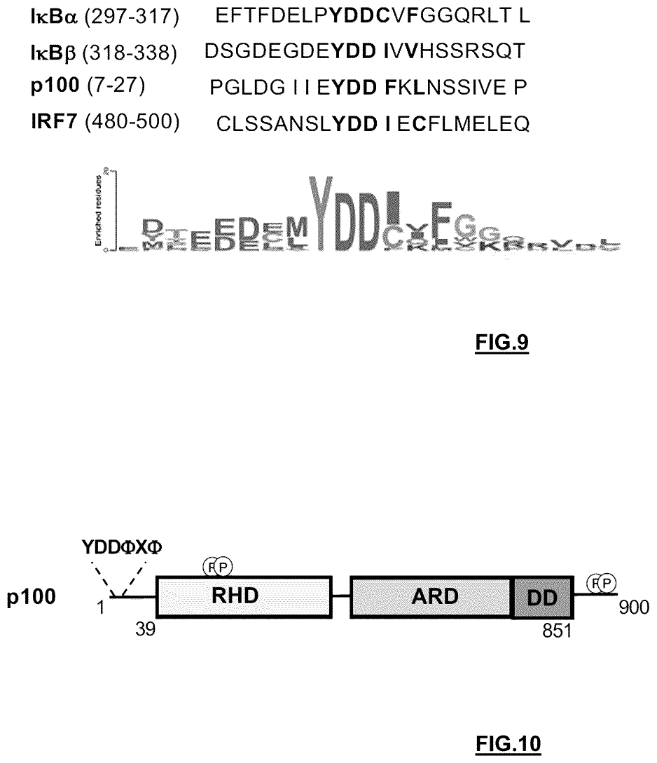

Proteome-wide bioinformatic analyses detected the strict YDDφXφ motif in three additional proteins, namely IκBβ, a canonical NF-κB substrate of IKKβ and orthologue of IκBα, p100, the alternative NF-κB substrate of IKKα and interferon regulatory factor 7 (IRF7), which is also a substrate of IKKα (FIG. 9). Peptides comprising the YDDφXφ sequences were designed from these proteins (referred to as IκBα (or P1), IκBβ pep, p100 pep, IRF7 pep, P2, P21, P2W, P3, P4 and P6 hereafter) and tested them against recombinant IKKα and IKKβ proteins by pulldown. Results show that IKKβ binds uniquely to peptides derived from the catalytic IκB substrates, and not to p100 and IRF7 peptides (FIG. 13A, right panel). In contrast, IKKα interacts with all peptides tested (FIG. 13A, left panel; FIG. 13B).

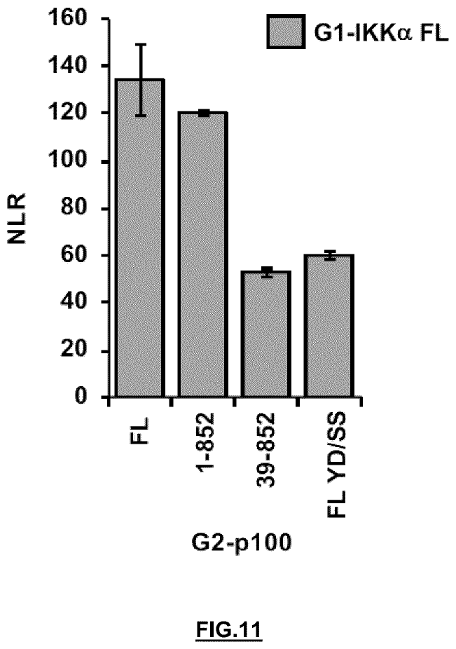

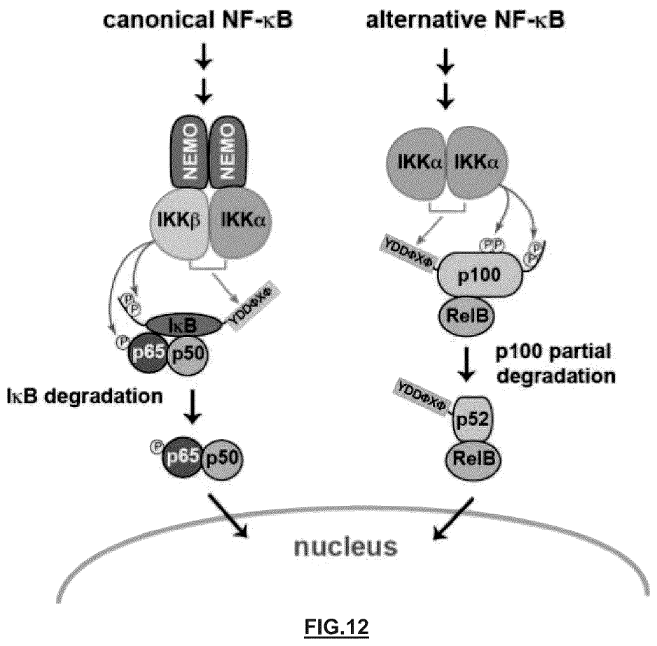

The alternative p100 substrate consists of an N-terminal disordered region, followed by a Rel homology domain (RHD), an ankyrin repeat domain (ARD), a death domain (DD) and a disordered C-terminal region (residues 852-900) (FIG. 10). The YDDφXφ motif is located in the N-terminal disordered region, whereas the IKKα phosphorylation sites are situated within the RHD and C-terminal regions. p100 pep binds to purified IKKα with an equilibrium affinity constant (K_{D}=11 μM) that is very similar to that of IκBα pep (9 μM). In vivo GPCA analyses show that deletion of the C-terminal region of p100 harboring some of the phospho-acceptor residues does not affect IKKα interaction with p100 (FIG. 11, 1 compare FL with 1-852). Instead, a double Ser substitution at the Y₁ and D₃ positions of the docking motif (i.e. Y15S/D17S) or deletion of the motif strongly reduces binding to IKKα (FIG. 11, compare FL with FL YD/SS and 39-852).

In IRF7, the YDDφXφ motif is located in the C-terminal disordered region, just downstream of the serine cluster phosphorylated by the TBK1/IKKε kinase regulating the interferon pathway. While, the inventors were able to reproduce the interaction between full length IRF7 and IKKα proteins by GPCA, this interaction does not appear to be affected by mutation of the Y₁ and D₃ motif positions of IRF7. Consistently, purified IKKα displays a low affinity for the synthetic IRF7 pep (K_{D}>90 μM), which could explain the marginal impact of the YDDφXφ motif in the context of IRF7 recruitment to IKKα. To investigate whether phosphorylation of IRF7 could modulate the motif contributions, the inventors introduced multiple phosphomimetic substitutions at serines residues within the cluster targeted by TBK1/IKKε and tested the interactions with IKKα. Results show no difference between the wt and phosphomimetic IRF7 constructs.

Taken together, these results show that the YDDφXφ motif is required for docking of IKKα and IKKβ homo- and heterodimers to substrates of the canonical (IκB) and alternative (p100) NF-κB pathways.

### CONCLUSION

The inventors shows that IKKs have developed an original strategy, which consist of SLIM docking to a groove at the interface of SDD domain dimers. Since dimerization is also necessary for kinase activation through the formation of higher order oligomers, it appears that the SDD domain has evolved as a versatile structure, which ensures kinase activation and, at the same time, interaction with the substrate. Interestingly, their results indicate that the IKKα subunit has higher affinity for the YDDφXφ motif as compared to the IKKβ subunit. This suggests that, in the canonical complex, in addition to the contributions related to IKKβ activation, IKKα has a second function, which consists of providing docking affinity for substrate recruitment. At the same time, this also suggests that this higher docking affinity of IKKα could compensate for the absence of NEMO in the alternative IKK complex. Inventors provide evidence for the requirement of the YDDφXφ motif for recruitment of NF-κB substrates IκB and p100 to IKKs. Deregulation of NF-κB signaling occurs in many pathologies, including infectious, inflammatory and autoimmune diseases, and cancer. Many enzymatic inhibitors of IKKβ have been developed, which lead to numerous side effects. Peptide inhibitors derived from the NEMO subunit, which inhibit the canonical pathway by targeting the NEMO-IKKα/β or NEMO-polyUb interfaces have been proposed as alternative therapeutic approaches. Here, the inventors show that a bivalent YDDφXφ peptide efficiently blocks canonical NFκB signaling. Given the requirement for the YDDφXφ motif in the IKKα/p100 interaction, the inventors expect this peptide to also have an inhibitory effect on the alternative NF-κB pathway. To conclude, this work unveils the mechanisms substrate recruitment by IKKs in NF-κB signaling, thus providing a rational framework for the design of future therapeutic approaches.

1. An isolated peptide having 10 to 35 amino acids comprising the amino acid sequence YDDφ₁Xφ₂, wherein φ₁ and φ₂ are hydrophobic amino acids and X is any amino acid.

2. The peptide according to claim 1, wherein said peptide binds to IKK dimers.

3. The peptide according to claim 1, wherein φ₁ is leucine or phenylalanine.

4. The peptide according to claim 1, wherein said peptide comprises a sequence with at least 75% of identity with:
the amino acid sequence EFTEDELPYDDCVFGGQRLTL (SEQ ID NO: 1), or
the amino acid sequence GLDGIIEYDDFKLNSSIVEPKE (SEQ ID NO: 2).

5. The peptide according to claim 1, wherein said peptide comprises the amino acid sequence EFTEDELPYDDLVFGGQRLTL (SEQ ID NO: 3).

6. A polypeptide fusion construct comprising a tag fused to the N-terminal end of a peptide according to claim 1.

7. The polypeptide fusion construct of claim 6, wherein the tag is the E3 peptide of sequence SEQ ID NO: 8.

8. A method of inhibiting the NF-κB signaling pathway comprising contacting in vitro IKKα and/or IKKβ with a peptide according to claim 1 or a polypeptide fusion construct.

9. A method of inhibiting the NF-κB signaling pathway comprising contacting in cellulo a polypeptide fusion construct according to claim 6.

10. A pharmaceutical composition comprising a peptide as defined in claim 1, or a polypeptide fusion construct comprising a tag fused to the N-terminal end of the peptide.

11. The pharmaceutical composition according to claim 10 for use in a method for treating and/or preventing a disorder regulated by the NF-κB signaling pathway in a subject in need thereof.

12. The pharmaceutical composition for use according to claim 11 in a method for wherein said disorder is linked to an hyperactivation of the NF-κB signaling pathway.

13. The pharmaceutical composition for use according to claim 11, wherein said disorder regulated by the NF-κB signaling pathway is selected from the group consisting of inflammatory diseases, oncogenesis, autoimmune diseases and viral infection.

14. The pharmaceutical composition for use according to claim 10, wherein said disorder is selected from the group consisting of rheumatoid arthritis, inflammatory bowel disease, multiple sclerosis, lymphomas, multiple myeloma and colorectal cancer.

15. The pharmaceutical composition for use according to claim 10, wherein the pharmaceutical composition is formulated for a parenteral, intravenous, intradermal, intracerebroventricular, subcutaneous, intramuscular, intraperitoneal, oral, intradermal, transdermal, transmucosal or intraocular administration.

16. A method of identifying small molecules targeting the docking groove of IKK dimers, the method comprising contacting IKK dimers with a candidate small molecule and a peptide according to claim 1 or a polypeptide fusion construct comprising a tag fused to the N-terminal end of the peptide.

17. The method according to claim 16 wherein the peptide or the polypeptide fusion construct further comprises a peptide tag.

18. The method according to claim 16 wherein the peptide or the polypeptide fusion construct is labeled with a fluorophore.

19. An isolated nucleic acid encoding a peptide according to claim 1 or a polypeptide fusion construct comprising a tag fused to the N-terminal end of the peptide.

20. A vector comprising a nucleic acid according to claim 19.

21. A host cell comprising a nucleic acid according to claim 19 or a vector comprising the nucleic acid.
